(19) United States
(12) Patent Application Publication
(10) Pub. No.: US 2018/0161579 A1
(43) Pub. Date: Jun. 14, 2018

(54) EXTRANASAL STIMULATION DEVICES AND METHODS

(71) Applicant: Oculeve, Inc., South San Francisco, CA (US)

(72) Inventors: Manfred FRANKE, Valencia, CA (US); Chao LIU, San Francisco, CA (US); Douglas Michael ACKERMANN, Reno, NV (US); Diane Michelle SENCHYNA, Corona del Mar, CA (US); James Donald LOUDIN, Alhambra, CA (US)

(21) Appl. No.: 15/829,847

(22) Filed: Dec. 1, 2017

Related U.S. Application Data
(60) Provisional application No. 62/429,065, filed on Dec. 1, 2016.

(51) Int. Cl. A61N 1/36

(52) U.S. Cl. CPC A61N 1/36046; A61F 9/04; A61N 1/36014

(57) ABSTRACT

A stimulation system for treating a condition (e.g., dry eye, Meibomian gland disease) of a subject includes a stimulator configured to deliver a stimulus to external facial tissue of the subject, at least one sensor configured to detect a characteristic of the subject, and a control system in communication with the sensor and configured to adjust the stimulus at least partially based on the detected subject characteristic. In some variations, the control system may be configured to adjust the stimulus at least partially based on a detected environmental condition. The stimulator may be configured to activate a nerve of the subject, thereby increasing tear production. The stimulation system may, for example, include a device configured to be worn by a subject such as eyeglasses or a mask, or held by a subject such as handheld stimulator device.

### CROSS-REFERENCE TO RELATED APPLICATIONS

This application claims priority to U.S. Patent Application Ser. No. 62/429,065, filed on Dec. 1, 2016, which is incorporated herein in its entirety by this reference.

### TECHNICAL FIELD

This invention relates generally to extranasal stimulation devices and methods to relieve symptoms associated with various conditions such as dry eye, contact lens discomfort, blepharitis, Meibomian gland dysfunction, and headache.

### BACKGROUND

Dry Eye Disease (“DED”) is a condition that affects millions of people worldwide. More than 40 million people in North America have some form of dry eye, and many millions more suffer worldwide. DED results from the disruption of the natural tear film on the surface of the eye, and can result in ocular discomfort, visual disturbance, and a reduction in vision-related quality of life. Activities of daily living such as driving, computer use, housework, and reading have also been shown to be negatively impacted by DED. Patients with severe cases of DED are at risk for serious ocular health deficiencies such as corneal ulceration, and can experience a quality of life deficiency comparable to that of moderate-severe angina.

DED is progressive in nature, and fundamentally results from insufficient tear coverage on the surface of the eye. This poor tear coverage prevents healthy gas exchange and nutrient transport for the ocular surface, promotes cellular desiccation and creates a poor refractive surface for vision. Poor tear coverage typically results from: 1) insufficient aqueous tear production from the lacrimal glands (e.g. secondary to post-menopausal hormonal deficiency, auto-immune disease, LASIK surgery, etc.), and/or 2) excessive evaporation of aqueous tear resulting from dysfunction of the Meibomian glands. Low tear volume causes a hyperosmolar environment that induces an inflamed state of the ocular surface. This inflammatory response induces apoptosis of the surface cells which in turn prevents proper distribution of the tear film on the ocular surface so that any given tear volume is rendered less effective. This initiates a vicious cycle where more inflammation can ensue causing more surface cell damage, etc. Additionally, the neural control loop, which controls reflex tear activation, is disrupted because the sensory neurons in the surface of the eye are damaged. As a result, fewer tears are secreted and a second vicious cycle develops that results in further progression of the disease (fewer tears cause nerve cell loss, which results in fewer tears, etc.).

There is a wide spectrum of treatments for DED. Treatment options include artificial tear substitutes, topical cyclosporine, omega-3 fatty acid supplements, punctal plugs and moisture chamber goggles. Patients with severe disease may further be treated with punctal cautery, systemic cholinergic agonists, systemic anti-inflammatory agents, mucolytic agents, autologous serum tears, PROSE scleral contact lenses and tarsorrhaphy. However, many of these existing treatment options are relatively invasive and/or cumbersome for a patient to use. Accordingly, it would be desirable to have an improved treatment for dry eye and other ocular conditions.

### SUMMARY

Generally, described herein are devices and methods for extranasal stimulation to treat a condition of a subject, such as dry eye, contact lens discomfort, blepharitis, Meibomian gland dysfunction, headache, etc.

In some variations, a system for treating a condition of a subject may include a stimulator configured to deliver a stimulus to external facial tissue of the subject, at least one sensor configured to detect a characteristic of the subject, and a control system in communication with the sensor and configured to adjust the stimulus at least partially based on the detected characteristic of the subject. The stimulus may activate a nerve of the subject, thereby increasing tear production. Exemplary nerves for activation include one or more branches of the ophthalmic nerve (CN VI) and the facial nerve. Additionally or alternatively, in some variations, a stimulus may energize or activate one or more facial muscles, such as the orbicularis muscle.

Similarly, in some variations, a stimulation method for treating a condition of a subject may include delivering a stimulus to external facial tissue of the subject such that the stimulus activates a nerve of the subject, thereby increasing tear production; and adjusting the stimulus at least partially based on a detected characteristic of the subject.

In some variations of the systems and methods described herein, the stimulus may include electrical stimulation, such as from a hydrogel electrode or other suitable electrode. In other variations, the stimulus may additionally or alternatively include ultrasound stimulation, such as from an ultrasound transducer. Other suitable forms of stimulation may additionally or alternatively be provided.

One or more various sensors may be configured to be detect characteristics of a subject, such as one or more symptoms of dry eye. For example, the stimulation system may include an image sensor configured to image an ocular region of the subject, and/or an electromyography sensor configured to detect facial muscle contractions. Such sensors may be configured to detect, for example, blinking data (e.g., blink rate, blink duration, and/or blink strength), eye redness, tear meniscus height, temperature of an ocular region of the subject, an indication of successful stimulation for tear production, etc.

Additionally or alternatively, in some variations, the stimulation system may include a second sensor configured to detect an environmental condition. For example, the second sensor may be configured to measure ambient light, wind, humidity, and the like which may tend to exacerbate dry eye symptoms for the subject. The control system may be further configured to adjust the stimulus at least partially based on the detected environmental condition.

In some variations, adjustment of the stimulus may be performed at least partially based on at least one of a detected characteristic of the subject and a detected environmental condition. Additionally or alternatively, adjustment of the stimulus may be performed in response to input of the subject.

Furthermore, generally, a stimulation system may include various form factors. For example, in one variation, a system for treating a condition of a subject may include eyeglasses configured to be worn by the subject, and at least one stimulator coupled to the eyeglasses and configured to deliver a stimulus to external facial tissue of the subject. In some variations, the stimulator may be disposed on a nosepad of the eyeglasses, while in other variations the stimulator may additionally or alternatively be disposed on a nasal strip coupled to the eyeglasses. The stimulus may include, for example, electrical stimulation and/or ultrasound stimulation. The stimulus may be configured to activate a nerve of the subject, thereby increasing tear production.

As another example, a system for treating a condition of a subject may include a mask configured to be worn by the subject, wherein at least a portion of the mask is conformable to external facial tissue of the subject, and at least one stimulator coupled to the mask and configured to deliver a stimulus to the external facial tissue of the subject. In some variations, the system may further include one or more heat sources coupled to the mask. The stimulus may include, for example, electrical stimulation and/or ultrasound stimulation. The stimulus may be configured to activate a nerve of the subject, thereby increasing tear production. Furthermore, in some variations, the system may include a plurality of stimulators arranged around an ocular region of the mask, such that the stimulators are configured to stimulate external facial tissue at least partially around an orbit of the subject. Such an arrangement may, for example, strengthen an orbicularis muscle of the subject.

As yet another example, a system for treating a conditions of a subject may include a handheld body, a projection coupled to the body where at least a portion of the projection is configured to conform to external facial tissue of the subject, and at least one stimulator coupled to the projection and configured to deliver a stimulus to the external facial tissue of the subject. For example, in some variations, the projection may include a concave surface, and the stimulator may be coupled to a tissue-facing side of the concave surface for stimulating the subject. Additionally, the system may include a second stimulator coupled to the tissue-facing side of the concave surface. The stimulus may include, for example, electrical stimulation and/or ultrasound stimulation. The stimulus may be configured to activate a nerve of the subject, thereby increasing tear production. In some variations, the projection may be removable, or reversibly attachable, to the handheld body.

Also described herein are other examples of stimulation systems for providing extranasal systems and carrying about the described methods, such as goggles, eyelid pads, over-the-ear stimulators such as a clip or earmuffs, and the like.

### BRIEF DESCRIPTION OF THE DRAWINGS

FIG. 1 is a schematic illustration of a stimulation device for treating a condition of a subject.

FIG. 2 is a graphical illustration of various nerves, including branches of the ophthalmic nerve (CN VI) on or near the face as potential targets for extranasal stimulation to treat a condition of a subject.

FIGS. 3A and 3B are front and rear views, respectively, of an exemplary eyeglasses variation of a stimulation device.

FIG. 4 is a schematic illustration of an exemplary goggles variation of a stimulation device.

FIG. 5 is a schematic illustration of an exemplary eye mask variation of a stimulation device.

FIG. 6 is a schematic illustration of another exemplary eye mask variation of a stimulation device.

FIGS. 7A and 7B are front and rear views, respectively, of an exemplary full face mask variation of a stimulation device.

FIGS. 8A and 8B are front and rear views, respectively, of another exemplary face mask variation of a stimulation device.

FIG. 9A is a schematic illustration of an exemplary eyelid pad variation of a stimulation device with a heating source. FIG. 9B is a schematic illustration of an exemplary eyelid pad variation of a stimulation device with a plurality of stimulators.

FIG. 10 is a schematic illustration of an exemplary eyeglasses variation of a stimulation device including an external nasal strip.

FIGS. 11A-11C are perspective, cut-away rear, and cut-away side views, respectively, of an exemplary handheld variation of a stimulation device. FIG. 11D is a partially disassembled view of the stimulation device depicted in FIGS. 11A-11C, along with a cover and a charge station.

FIGS. 11E and 11F are exemplary illustrations of use of the stimulation device depicted in FIGS. 11A-11C.

FIGS. 12A-12C are side, front, and rear views, respectively, of another exemplary handheld variation of a stimulation device. FIG. 12D is a partially disassembled view of the stimulation device depicted in FIGS. 12A-12C.

FIG. 13 is schematic illustration of another exemplary eyeglasses variation of a stimulation device.

FIGS. 14A and 14B are perspective and underside views, respectively, of an exemplary over-the-ear variation of a stimulation device.

FIG. 15A is a perspective view of an exemplary earmuff variation of a stimulation device.

FIG. 15B is a detailed view of an interior of an earmuff pad of the stimulation device depicted in FIG. 15A.

FIG. 16 is a schematic illustration of one variation of a method for treating a condition of a subject.

FIG. 17 is a plot illustrating decrease in matrix metalloproteinase 9 (MMP-9) concentration in tear film in subjects compared to that in control subjects, following an exemplary application of extranasal stimulation as described in Example 2.

FIG. 18 is a plot illustrating decrease in interleukin-8 (IL-8) concentration in tear film in subjects compared to that in control subjects, following an exemplary application of extranasal stimulation as described in Example 2.

### DETAILED DESCRIPTION

Non-limiting examples of various aspects and variations of the invention are described herein and illustrated in the accompanying drawings.

Described herein are stimulation devices and methods to relieve symptoms associated with conditions such as dry eye, contact lens discomfort, blepharitis, Meibomian gland dysfunction, ocular discomfort due to wearing contact lenses, and headache (e.g., sinus headache). For example, at least some of the stimulation devices and methods described herein may be used to increase tear production (increase in tear and/or tear component release). As another example, at least some of the stimulation devices and methods described herein may be used to retrain certain facial muscles to improve tear and/or tear component release, such as stimulation of the orbicularis muscle to improve expression of the tear component meibum. As yet another example, at least some of the stimulation devices and methods described herein may be used to improve airway passages, such as to aid a subject or patient having breathing problems (e.g., congestion), sinus headaches, etc. These and other applications of the stimulation devices and methods are described in further detail below.

### Stimulation Devices

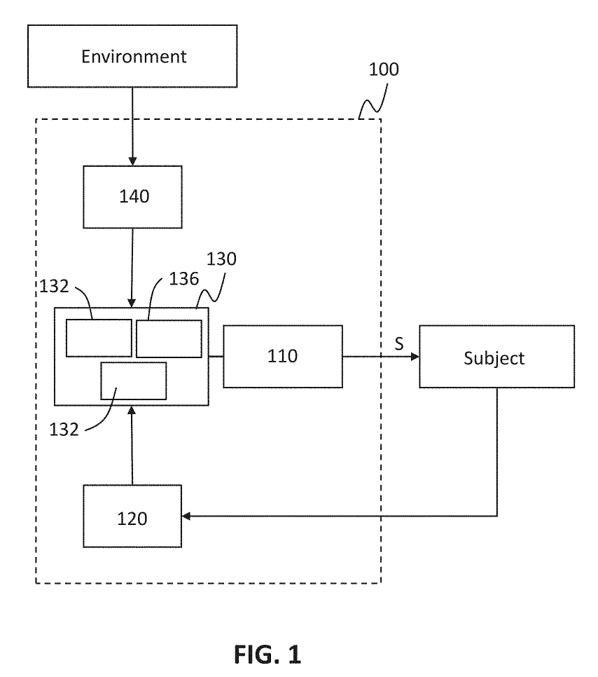

As shown in the schematic of FIG. 1, in some variations, a stimulation device 100 for treating a condition of a subject may include a stimulator 110 configured to deliver a stimulus (“S”) to external tissue of the subject such that the stimulus activates a nerve of the subject, at least one sensor 120 configured to detect a characteristic of the subject, and a control system 130 in communication with the sensor and configured to adjust the stimulus at least partially based on the detected characteristic of the subject. In some variations, as further described below, the stimulation device 100 may additionally or alternatively include at least one sensor 140 configured to detect an environmental condition. The control system 130 may be configured to adjust the stimulus at least partially based on the detected environmental characteristic.

The one or more sensors (e.g., sensors 120 and/or 140) in the stimulation device may, for example, enable real-time or substantially real-time feedback regarding a condition of the subject and/or the environment that may be used by the control system 130 to trigger and/or modify stimulation. For example, as further described below, the one or more sensors may provide data that may be used to determine whether the subject would benefit from treatment via stimulation, and/or to determine mode of stimulation (e.g., electrical, ultrasound, etc.) intensity, duration, particular stimulation pattern, location of stimulation, and/or other suitable parameters of the stimulation. As another example, the one or more sensors in the stimulation device may provide data that may be used to assess efficacy of the stimulation treatment, and/or to determine whether further adjustments or modifications to the stimulation would be beneficial to better treat the subject. Such triggering and/or modification of the stimulation to the subject may be automatically adjusted. Additionally or alternatively, the stimulus may be controlled by the subject (or other user), such as based on a manual input through a user interface.

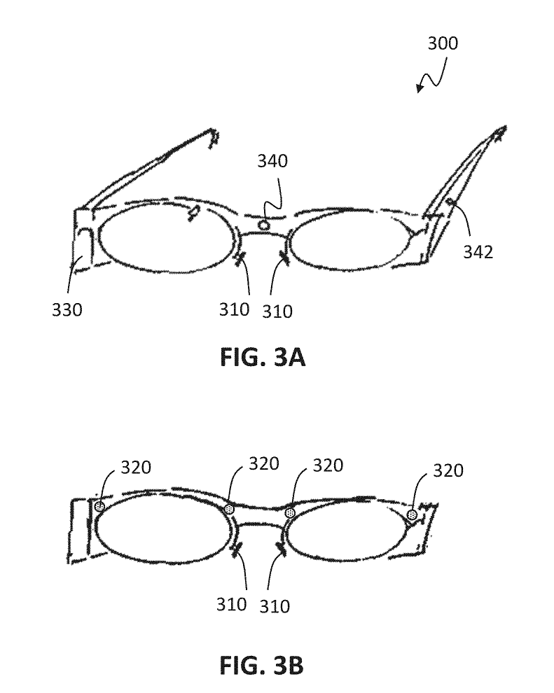

Different variations of the stimulation device 100 may be of any suitable form factor for positioning the stimulator at an appropriate location for delivering the stimulus to the subject. FIGS. 3A-15B depict exemplary variations of stimulation devices. For example, as further described below, in some variations at least a portion of the stimulation device may be part of a wearable device (e.g., eyeglasses, goggles, mask, adhesive pads, nose strips, external nose clips, nose rings, etc.) that positions the stimulator to be adjacent a suitable target on the subject when the wearable device is worn by the subject. As another example, as further described below, the stimulation device may be a handheld device that can be held by the subject (or other user, such as a medical professional treating the subject) against his or her face to position the stimulator adjacent to a suitable target.

In some variations, the form factor of the device may be commonplace and/or discreet, such that stimulation to tissue of the subject may be delivered in a manner that is less likely to be noticed by others, cause embarrassment, etc. Accordingly, a subject may more easily and more frequently obtain relief, via the stimulation, for one or more conditions such as those described herein.

### Stimulator

The stimulation device 100 may include one or more stimulator components for stimulating a nerve or other external tissue (e.g., extranasal tissue) of a subject with one or more different kinds of stimulation. For example, as further described below, the stimulation device may include one or more electrodes providing electrical stimulation, one or more transducers providing ultrasonic or other acoustic stimulation, or other suitable forms of stimulation (e.g., chemical, heat, etc.). Stimulation waveforms delivered by the stimulators described herein may be tailored for specific treatment regimens and/or specific patients.

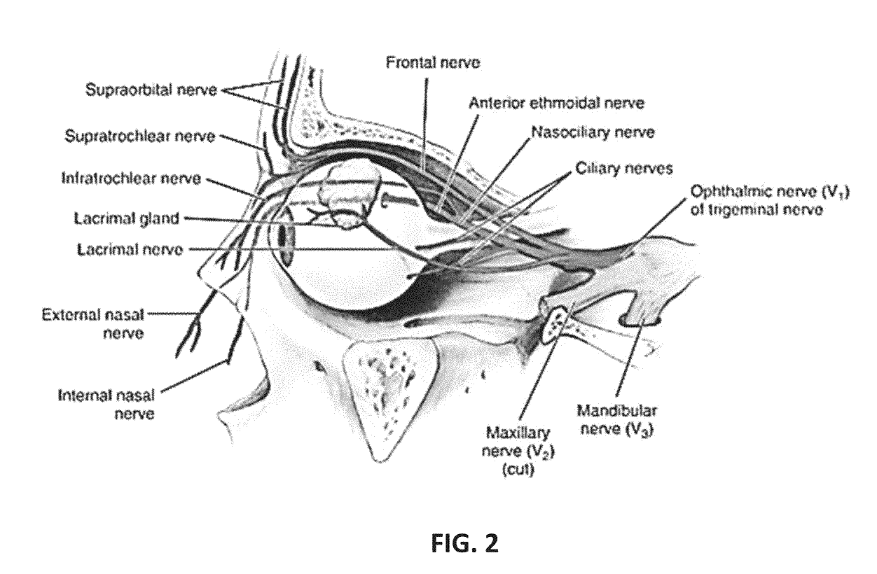

The one or more stimulators may be located on the stimulation device to target suitable regions on or near a user's face. Suitable targets for stimulation include, for example, nerves and/or muscles of the eyelids, near the eyelids, the eyebrows, the nose between the eyes (e.g., nose bridge), and the nose below the eyeline. FIG. 2 is a graphical illustration of various nerves, including branches of the ophthalmic nerve (CN VI) on or near the face. Many of these nerves may be suitable targets for external stimulation with a stimulator device such as any of those described herein, for treatment of ocular conditions and/or other conditions such as dry eye, blepharitis, and Meibomian gland disease. In some variations, the ophthalmic nerve (CN VI) branches of the face may include suitable stimulation targets. For example, in some variations, the stimulation targets may include the infratrochlear nerve and the anterior ethmoidal nerve on the outside of the nose. As another example, the stimulation targets nay include the lacrimal nerve (innervating the lateral eyelids), and the supratrochlear nerve and supratrochlear nerve (innervating the upper eyelid). Furthermore, in some variations, suitable targets for stimulation include the exit point of the facial nerve (CN VII), in the region between the nose and the ear along the side of the face. Stimulation of such anatomical targets described herein may lead to an increase in tear and/or tear component release.

In some variations, one or more stimulators may be located on the stimulation device to target and strengthen the orbicularis muscle located around the orbit of the eye. For example, a plurality of stimulators (e.g., electrical, ultrasound, etc.) may be arranged so as to stimulate an area around the orbit of the subject's face. The orbicularis muscle is the muscle that closes the eyelids. With age and/or muscular conditions, the orbicularis muscle may atrophy and make expression of meibum (a tear component) more difficult. By targeting stimulation on the orbicularis muscle, some variations of a stimulation device may strengthen and regrow the muscle for easier meibum expression. Furthermore, in some variations, stimulation of the orbicularis muscle may provide cosmetic benefits such as improved facial appearance, as the result of eyelids gaining a healthier appearance in shape and strength.

In some variations, the stimulator 110 may include at least one electrode for providing electrical stimulation to an anatomical target. The electrode may be coupled to a support structure so as to contact target tissue of the subject. For example, in the exemplary variation of an eyeglasses stimulation device 300 shown in FIGS. 3A and 3B, one or more stimulators 310 may include electrodes coupled to a nosepad region of the eyeglasses, such that when the eyeglasses stimulation device 300 is worn by a user, the stimulators 310 contact the external tissue of the user's nose. In this variation, the stimulators 310 may, for example, deliver a stimulus to the exit point of the anterior ethmoidal nerve and/or the infratrochlear nerve (and/or other facial anatomy near the eyes). Other exemplary locations of the stimulator on the stimulation device are illustrated and described below with reference to FIGS. 3A-15B. However, the one or more stimulators may be coupled to the stimulation device in any suitable manner, in any suitable location on the stimulation device.

The stimulator 110 may, in some variations, be configured to deliver patterned stimulation waveforms (e.g., electrical stimulation) to an anatomical structure as described in U.S. Pat. No. 9,687,652 titled “STIMULATION PATTERNS FOR TREATING DRY EYE” and filed Jul. 24, 2015, which is incorporated herein in its entirety by this reference.

When patterning of stimulation waveforms is employed, waveform parameters such as the shape, the frequency, the amplitude, and the pulse width may be modulated. The frequency, pulse-width, and/or amplitude of the waveform may be modulated linearly, exponentially, as a sawtooth, a sinusoidal form, etc., or they may be modulated randomly. The stimulation can also be interrupted as part of the patterning. That is, the stimulation can be in an on/off condition, e.g., for durations of 1 second on/1 second off, 5 seconds on/5 seconds off, etc. Modulation of the waveform shape (e.g., rectangular vs. triangular vs. exponential) in a rhythmic or non-deterministic, non-rhythmic fashion may also be used. Thus, numerous variations in waveform patterning can be achieved. It should be understood that combinations of these parameter changes over time in a repetitive manner may also be considered patterning. In some instances, random patterning may be employed. Patterning may help to prevent subject habituation to the applied stimulation (i.e., may help to prevent the subject response to the stimulation decreasing during stimulation).

The stimulation may be delivered periodically at regular or irregular intervals. Stimulation bursts may be delivered periodically at regular or irregular intervals. The stimulation amplitude, pulse width, or frequency may be modified during the course of stimulation. For example, the stimulation amplitude may be ramped from a low amplitude to a higher amplitude over a period of time. In other variations, the stimulation amplitude may be ramped from a high amplitude to a lower amplitude over a period of time. The stimulation pulse width may also be ramped from a low pulse width to a higher pulse width over a period of time. The stimulation pulse width may be ramped from a high pulse width to a lower pulse width over a period of time. The ramp period may be between 1 second and 15 minutes. Alternatively, the ramp period may be between 5 seconds and 30 seconds.

It should be appreciated any of the above waveform parameters and variations in parameters may be combined to generate a patterned stimulation waveform, and these waveforms may be delivered by any of the stimulators described herein. For example, in variations where the stimulation is electrical and comprises a biphasic pulse, the biphasic pulse may have any suitable frequencies, pulse widths, and amplitudes. The stimulation amplitude, pulse width, and frequency may be the same from pulse to pulse, or may vary over time, as described in more detail herein. Combinations of these parameters may increase the efficacy and/or comfort of stimulation, and in some cases, the efficacy and/or comfort may differ by individual patient, as described in more detail herein. Exemplary patterned waveform parameters for extranasal electrical stimulators are listed below in Table 1.

TABLE 1
Exemplary Waveform Parameters

| | Waveform Parameters | | | | |
|---|---|---|---|---|---|
| | | | Fre- | Pulse | Am- |
| Device | Stimulation | | quency | Width | plitude |
| Type | Target | On/Off | (Hz) | (PW) | (mA) |
| Nasal | Internal and | Constant on | 30 | 0 μs to | 0.1 to 10 |
| Stimulator | external | Constant on | 50 | 1000 μs | |
| | nasal | Constant on | 80 | | |
| | nerves | Constant on | 150 | | |
| | (e.g., | 1 sec on/ | 30 | | |
| | anterior | 1 sec off | | | |
| | ethmoidal | 1 sec on/ | 50 | | |
| | nerve) | 1 sec off | | | |
| | | 1 sec on/ | 80 | | |
| | | 1 sec off | | | |
| | | Constant on | 30 | | |
| | | 1 sec on/ | 70 | | |
| | | 1 sec off | | | |

It should be appreciated that electrical stimulation waveforms may be delivered via a multi-polar, such as bipolar, tripolar, quad-polar, or higher-polar configuration or a monopolar configuration with distal return. The waveforms may be a sinusoidal, quasi-sinusoidal, square-wave, sawtooth, ramped, or triangular waveforms, truncated-versions thereof (e.g., where the waveform plateaus when a certain amplitude is reached), or the like.

In variations in which electrical stimulation includes an alternating monophasic pulsed waveform, each pulse delivered by the stimulator may have a single phase, and successive pulses may have alternating polarities. Generally, the alternating monophasic pulses are delivered in pairs at a given frequency (such as one or more of the frequencies listed above, such as between 30 Hz and 80 Hz), and may have an inter-pulse interval between the first and second pulse of the pair (e.g., about 100 μs, between 50 μs and 150 μs or the like). Each pulse may be current-controlled or voltage-controlled, and consecutive pulses need not be both current-controlled or both voltage-controlled. In some variations where the pulse waveform is charged-balanced, the waveform may comprise a passive charge-balancing phase after delivery of a pair of monophasic pulses, which may allow the waveform to compensate for charge differences between the pulses.

When a stimulator configured to deliver an electrical stimulation waveform is positioned to place an electrode on either side of the nose, alternating monophasic pulses may promote bilateral stimulation of nasal tissue. The pulses of a first phase may stimulate a first side of the nose (while providing a charge-balancing phase to a second side of the nose), while the pulses of the opposite phase may stimulate the second side of the nose (while providing a charge-balancing phase to the first side of the nose), since nerves may respond differently to anodic and cathodic pulses. The inter-pulse interval may give time for the stimulation provided by a first phase pulse to activate/polarize the target nerves prior to being reversed by an opposite phase pulse.

In variations configured to deliver electrical stimulation, the stimulator may include one or more conductive materials such as metal (e.g., stainless steel, titanium, tantalum, platinum or platinum-iridium, other allows thereof, or the like), conductive ceramics (e.g., titanium nitride), liquids, and/or gels, etc. As another example, the electrode may include an electrically conductive hydrogel. The hydrogel may include any suitable hydrogel, such as those described in U.S. Pat. No. 9,770,583 titled “POLYMER FORMULATIONS FOR NASOLACRIMAL STIMULATION” filed Feb. 24, 2015, which is incorporated herein in its entirety by this reference. The conductive material of the stimulator may be coupled to stimulator circuitry and/or other aspects of the control system via one or more conductive leads (e.g., wires) or conductive traces.

Additionally or alternatively, ultrasonic energy may be delivered to external tissue by a stimulator comprising one or more ultrasound transducers. For example, one or more pulses of air may be delivered to stimulate tissue. The pulses of air may be generated via a source of compressed gas (e.g., air), or the like. In some variations, the gas may be warmed or cooled (e.g., mechanically or via one or more thermally-activated fibers). In some variations, the ultrasonic energy may be focused so as to concentrate the energy into a small focal zone in the target stimulation region. For example, the ultrasonic energy may be focused by an acoustic lens, a curved transducer, and/or a phased array, etc.

In other variations, one or more portions of a stimulator may be a heating source to provide thermal stimulation to tissue, such as with a resistive element that is activated to generate heat. Additionally or alternatively, one or more portions of a stimulator may include a cooling source (e.g., gel) to provide another form of thermal stimulation to tissue. In yet other variations, the stimulator may additionally or alternatively use one or more light-generating or magnetic field-generating elements to stimulate tissue. In yet other variations, a stimulator may include chemical stimulation (e.g., by releasing a chemical agent providing a chemical stimulus).

### Sensor

As shown in FIG. 1, the stimulation device 100 may include one or more sensors. For example, the stimulation device may include at least one sensor 120 configured to detect a characteristic of the subject, and/or at least one sensor 140 configured to detect an environmental condition. As described below, information detected by the sensors 120 and/or 140 may be used by the control system to adjust the stimulus from the stimulator 110.

Additionally or alternatively, information detected by the sensors 120 and/or 140 may be used to develop an adaptive learning algorithm that associates particular subject characteristics and/or environmental conditions with dry eye symptom severity (for an individual and/or for general populations). Other examples of adaptive algorithms are described below with respect to the control system.

### Sensors Detecting Subject Characteristics

Generally, in some variations, at least one sensor 120 may be configured to detect a characteristic of the subject relating to dry eye symptoms and/or symptoms of other ocular conditions. The control system may adjust the stimulation in response to this indication of dry eye symptoms.

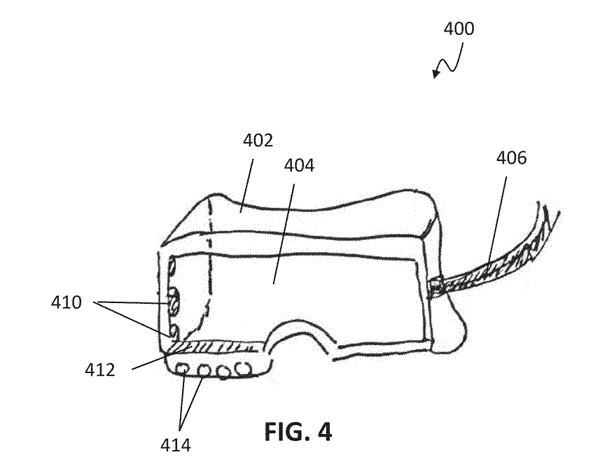

In some variations, the sensor 120 may include an image sensor. For example, in the exemplary variation of an eyeglasses stimulation device 300 shown in FIGS. 3A and 3B, at least one image sensor 320 (shown in FIG. 3B) may be disposed on an eye-facing surface of eyeglasses to be worn by the subject, or otherwise coupled to the eyeglasses to capture the eye of the subject within the image sensor's field of view. The image sensor 320 may include a camera for capturing still images and/or video, such as optical images, infrared (IR) images, etc. Although the image sensor 320 is described here as being coupled to eyeglasses, it should be understood that one or more image sensors 320 may similarly be disposed on other devices (e.g., devices as described below with reference to FIGS. 4-15B).

The image sensor 320 may be configured to detect, for example, one or more parameters related to blinking, such as blink rate, blink duration, and blink strength. An act of blinking may be determined based on detected movement of an upper eyelid and/or lower eyelid, and/or detected change in exposed surface area of an eye of the user. For example, such eyelid movement and/or change in exposed surface area of an eye may be determined using suitable machine vision techniques (e.g., edge detection). As another example, markers (e.g., IR markers) may be attached to facial skin (e.g., eyelids, eyebrows, etc.), and an IR camera system may track the movements of the markers to detect blinking parameters. Blinking data may be correlated to severity of dry eye symptoms. For example, in some variations, if the subject is blinking at a higher frequency, blinking for longer duration, and/or blinking more strongly, the subject may be experiencing more severe dry eye symptoms. Accordingly, blinking data may provide a basis for adjusting stimulation in order to provide the subject with suitable relief of dry eye symptoms. Similarly, blinking data may provide a basis for triggering stimulation that reminds the subject to blink when he or she has not blinked after a predetermined time or with at least a predetermined frequency. Stimulation for reminding the subject to blink may be similar to stimulation for increasing tear production, or may be specifically designed for reminder purposes (e.g., higher frequency, larger amplitude, shorter duration, etc. compared to stimulation for increasing tear production). Additionally or alternatively, the stimulation system may be configured to provide the subject with reminders to blink using other suitable mechanisms, such as vibration and/or audio.

Additionally or alternatively, the image sensor 320 may be configured to detect changes in blood vessels in the subject's eye. For example, an optical image from the image sensor may be analyzed via machine vision techniques applied to pixel colors, in order to measure eye redness of the subject. Eye redness may generally indicate forms of stress, irritation, etc. in the ocular region. For example, the intensity of localized redness, average redness of the subject's sclera, the pattern of redness across the surface of the eye, and/or other parameters relating to eye redness may be correlated to severity of dry eye symptoms. Accordingly, eye redness may provide a basis for adjusting stimulation in order to provide the subject with suitable relief of dry eye symptoms and other conditions.

As another example, the image sensor 320 may be configured to detect tear meniscus height (e.g., tear meniscus on the lower eyelid of the subject). For example, an image from the image sensor may be analyzed via machine vision techniques (e.g., edge detection) to measure height of the tear meniscus. In one exemplary embodiment, tear meniscus height may be measured as the distance between a tear meniscus and a lower eyelid of the subject (both detected, for example, using edge detection techniques on an optical image or a thermal image). Tear meniscus height may vary according to severity of dry eye in the subject. For example, if the user has a smaller tear meniscus, the subject may be experiencing more severe dry eye symptoms. Accordingly, tear meniscus height may provide a basis for adjusting stimulation in order to provide the subject with suitable relief of dry eye symptoms and other conditions.

As yet another example, the image sensor 320 may be used to measure tear film breakup time (TBUT) as an indication of dry eye symptoms. For example, with the subject not blinking, the image sensor 320 may monitor the tear film over time as evaporation causes the tear film to thin and eventually form dry spots due to insufficient wetting on the surface of the eye. The time that it takes for the first dry spot to form is referred to as the TBUT, where shorter TBUT times may be correlated to severity of dry eye symptoms. In some variations, as determining TBUT, dry spot formation may be identified with machine vision techniques applied to an image of the eye taken by the image sensor 320. For example, reflectivity or glossiness over the surface of the eye (e.g., as captured in an optical image by an optical camera) may be analyzed by applying machine vision techniques, and may be correlated to dry eye symptoms. In some variations, a fluorescine dye may be applied to the surface of the eye and mixed with tear film to better distinguish wet spots from dry spots. As another example, temperature distribution over the surface of the eye (e.g., as captured in a thermal image by an IR camera) may be analyzed and correlated to dry eye symptoms. Additionally or alternatively, TBUT may be determined by direct inspection (e.g., by a clinician) of the image and/or the subject's eye itself. Accordingly, tear film breakup time may provide a basis for adjusting stimulation in order to provide the subject with suitable relief of dry eye symptoms and other conditions.

As another example, the image sensor may be configured to assess temperature of the ocular region, such as the cornea, conjunctive, and lower and/or upper eyelid. Temperature of any one or more areas of the ocular region may be measured, for example, using an IR image sensor generating a thermal image of the ocular region. Generally, a lower temperature in the ocular region (e.g., ocular surface) may correspond to decreased tear production (e.g., due to more rapid cooling observed in patients with dry eye symptoms compared to healthy patients). For example, in some variations, in response to detecting an ocular region (e.g., ocular surface) temperature that is below a predetermined threshold, and/or an ocular region (e.g., ocular surface) temperature drop that is greater than a predetermined threshold (e.g., between about 0.5 degrees Celsius and about 1.0 degree Celsius, or other suitable values), stimulation may be triggered or stimulation intensity may be increased. Accordingly, temperature of the ocular region may provide a basis for adjusting stimulation in order to provide the subject with suitable relief of dry eye symptoms and other conditions.

In some variations, the sensor 120 may include an electromyography (EMG) sensor configured to detect electrical activity produced by skeletal muscles. The EMG sensor may, for example, include an adhesive backing that allows the EMG sensor to couple to the skin of the subject proximate a muscle of interest. Alternatively, the EMG sensor may be coupled to a device (e.g., mask) that positions the EMG sensor against the skin of the subject. Leads from the EMG sensor, or another suitable communication scheme, may communicate signals from the EMG sensor to the control system for processing. For example, one or more EMG sensors may be disposed across the nose bridge of the subject and/or in the region between the subject's nose and ear, such that data from the EMG sensors may be used to determine whether the subject is blinking very strongly with his or her muscles in the nasal region. As described above, blink strength may be correlated to severity of dry eye symptoms. Accordingly, EMG data may provide a basis for adjusting stimulation in order to provide the subject with suitable relief of dry eye symptoms and other conditions.

As another example, one or more EMG sensors may be disposed along the jaw or cheek of the subject, such that data from the EMG sensors may be used to determine whether the subject is yawning. Since yawning often triggers tearing in healthy individual, EMG data may provide a basis for adjusting stimulation in order to provide the subject with appropriate tear production corresponding to the subject's yawning, particularly if the subject is experiencing symptoms of dry eye, Meibomian gland dysfunction, and/or blepharitis.

Additionally or alternatively, in some variations, at least one sensor 120 may be configured to detect a characteristic of the subject indicating successful stimulation. Accordingly, data from the sensor 120 may be used to confirm, for example, whether tear production has occurred. The control system may utilize this information as feedback to modify the stimulation to be delivered to the subject (if, for example, tear production has not occurred) until successful stimulation has been detected. Additionally or alternatively, the control system may maintain settings, such as frequency, pattern, intensity, and the like, for the stimulation delivered to the subject (if, for example, tear production has successfully occurred).

For example, in some variations, at least one sensor 120 may include an image sensor (e.g., similar to image sensor 320 as described above) configured to detect a dilation and constricting of the iris. For example, an image from the image sensor may be analyzed via machine vision techniques in order to determine whether the diameter of an iris of the subject's eye has increased and then decreased. The iris may open and close in response to stimulation. Accordingly, such fluctuation of the subject's iris may be an indication of successful stimulation.

Furthermore, any of the subject characteristics described above as being correlated to dry eye symptoms may also indicate successful stimulation, when such symptoms are reduced. For example, detection of reduced blink rate, reduced blink duration, and/or reduced blink strength (e.g., as detected by an image sensor and/or EMG sensor as described above, or other suitable sensor) may indicate successful stimulation to achieve increased tear production. As another example, decreased eye redness (e.g., as detected by an image sensor as described above) may indicate successful stimulation. As yet another example, increased tear meniscus height (e.g., as detected by an image sensor as described above), may indicate successful stimulation. Furthermore, an increase in temperature in the ocular region (e.g., as detected by an image sensor as described above) may be correlated to increased tear production and thus may indicate successful stimulation.

Although image sensors and EMG sensors are described above as detecting one or more characteristics such as blinking parameters, changes in blood vessels in the subject's eye, and tear meniscus height, it should be understood that these characteristics may be detected and measured with any suitable sensor arrangement. Furthermore, information from multiple sensors may be collected and compared in order to corroborate conclusions about severity of dry eye symptoms and/or success of stimulation, thereby improving accuracy of the assessment of the subject and improving the efficacy of the stimulation device for the subject.

### Sensors Detecting Environmental Conditions

Generally, in some variations, at least one sensor 140 may be configured to detect an environmental condition, such as an environmental condition that may cause or exacerbate dry eye symptoms. The control system may adjust the stimulation in response to such an environmental condition, such as to automatically trigger or modify the stimulation to increase tear production in the subject. Such stimulation may, for example, help provide relief of dry eye symptoms and/or pre-emptively reduce the likelihood of dry eye symptoms. Additionally, information from multiple sensors may be useful for developing more accurate adaptive algorithms for stimulation, as further described below.

For example, in the exemplary variation of an eyeglasses stimulation device shown in FIGS. 3A and 3B, the stimulation device may include at least one light sensor 340. The light sensor 340 may be outward-facing (facing away from the subject) when the subject wears the eyeglasses. For example, the light sensor 340 may be coupled to a nose bridge of the eyeglasses, though it may be coupled to the lens frame or other suitable portion of the eyeglasses. The light sensor 340 may, for example, detect whether an illuminated monitor such as a computer screen or television is facing the subject (e.g., indicating that the subject is likely staring at the illuminated monitor). For example, the light sensor 340 may detect a particular light intensity above a predetermined threshold, or a particular distribution of red, blue, and green wavelengths typical of an illuminated monitor, etc. As another example, the light sensor 340 may additionally or alternatively detect if such a computer screen has a refresh rate of about 50 Hz to about 100 Hz (e.g., based on strobe patterns of the detected light). Additionally or alternatively, an image sensor may detect or confirm the presence of a monitor and/or a screen refresh rate, based on pixel intensity or color distribution patterns typical of an illuminated monitor, etc. These environmental conditions may, at least in some individuals, tend to cause or exacerbate dry eye symptoms. Accordingly, the detected existence and/or nature of an illuminated monitor may provide a basis for adjustment of the stimulation in order to provide the subject with suitable relief and/or pre-emptive reduction of dry eye symptoms. Although the light sensor 340 is shown on an eyeglasses variation, it should be understood that such a light sensor may be disposed on other suitable devices, such as those described below with reference to FIGS. 4-15B.

Similarly, in some variations, the light sensor 340 may detect changes in light as the subject (while wearing the stimulation device 300) moves around and enters brightly-lit areas. For example, if detected light intensity exceeds a predetermined threshold, then the measurement may indicate that the subject has moved to a brighter environment. Similarly, a magnitude of change in detected light intensity that exceeds a predetermined threshold may indicated that the subject has moved to a brighter environment. When the subject moves to a brightly-lit area, he or she may experience photophobia or other forms of photosensitivity. Simulation by the stimulation device may, in some variations, reduce such photophobia. Accordingly, detected changes in light may provide a basis for adjusting stimulation in order to provide the subject with suitable relief and/or pre-emptive reduction of photophobia.

Additionally or alternatively, in some variations of a stimulation device including an image sensor, the light sensor 340 or another image sensor may provide data regarding ambient light so as to calibrate or normalize images from the image sensor. For example, the light sensor 340 may be used to subtract effects of ambient light on an optical image taken by the one or more image sensors 320. For example, reference pixel color values (e.g., red, green, and blue (RGB) intensity values) associated with ambient light may be determined from a reference image (e.g., an image taken of a reference surface with known characteristics). In order to compensate for effects of ambient light on an optical image of interest taken by the one or more image sensors 320, these reference pixel color values may be subtracted from the optical image of interest. However, other suitable manners of compensation for ambient light and/or other suitable calibration may be performed.

In other variations, other sensors 140 may be configured to detect other environmental conditions. For example, in the exemplary variation of an eyeglasses stimulation device shown in FIGS. 3A and 3B, another sensor 342 may additionally or alternatively be disposed on an arm of the eyeglasses, so to be near the temple of the subject when the subject is wearing the eyeglasses. However, the sensor 342 may be coupled to any suitable portion of the eyeglasses. Furthermore, although the sensor 342 is shown on an eyeglasses variation, it should be understood that such a sensor may be disposed on other kinds of devices, such as those described below with reference to FIGS. 4-15B.

In some variations, the sensor 342 may include a humidity sensor configured to detect ambient humidity. A low humidity environment may be correlated to an increased likelihood of dry eye symptoms and/or increased severity of dry eye symptoms. For example, low humidity may be detected when the subject is in an arid climate, in an air-conditioned or heated room or vehicle, etc. and may be at greater risk of experiencing dry eye symptoms. Accordingly, detected humidity may provide a basis for adjusting stimulation in order to provide the subject with suitable relief and/or preemptive reduction of dry eye symptoms.

The sensor 342 may additionally or alternatively include a pressure sensor configured to detect a windy environment. A windy environment may increase evaporation rate of tear film from a subject's eyes and increase likelihood and/or severity of dry eye symptoms. Accordingly, detected pressure correlated to wind may provide a basis for adjusting stimulation to relieve and/or preemptively reduce dry eye symptoms.

In some variations, the sensor 342 may include an accelerometer, an inertial measurement unit (IMU), a barometer, or other suitable sensor that may indicate when the subject is traveling in a type of vehicle that is often associated with an environment more likely to cause dry eye symptoms. For example, certain levels of acceleration and/or velocity measured by an accelerometer or IMU may, if exceeding a predetermined threshold, indicate that the subject is likely sitting in a car, bus, airplane, or other vehicle that often has a drier, air-conditioned environment. Similarly, a barometer may detect air pressure changes that may indicate that the subject is likely sitting in an airplane. As yet another example, an audio sensor may detect certain audio frequencies associated with plane travel, car travel, etc. Accordingly, detected acceleration, velocity, and/or air pressure changes, etc. may provide a basis for adjusting stimulation to relieve and/or preemptively reduce dry eye symptoms.

Although various sensors are described above as detecting one or more environmental conditions, it should be understood that these conditions may be detected and measured with any suitable sensor arrangement. Furthermore, information from multiple sensors may be collected and compared in order to corroborate conclusions about environmental conditions, thereby improving accuracy of the assessment of the environment and assessment of whether stimulation for tear production would be beneficial for the subject. Additionally, information from multiple sensors may be useful for developing more accurate adaptive algorithms for stimulation, as further described below.

### Control System and Electronics

Generally, the control system 130 may be configured to control a stimulus to be delivered to a subject via the stimulator. As shown in FIG. 1, the control system 130 may be in communication with one or more sensors (e.g., at least one sensor 120 detecting a characteristic of a subject, and/or at least one sensor 130 detecting an environmental condition). The control system 130 may be configured to adjust the stimulus delivered by the stimulator, where the adjustment is at least partially based on the detected subject characteristic.

The control system 130 may include circuitry and other suitable components configured to operate the stimulators and/or sensors as described herein. In some variations, the control system 130 may include a processor 132, memory 134, and/or a stimulation controller 136. The processor 132 may be configured to control operation of the various components of the control system 130 and/or analyze sensor data. For example, the processor 132 may be configured to receive data regarding one or more characteristics of the subject, and/or one or more environmental conditions and determine whether and how the stimulator will deliver stimulation the subject (e.g., as described above). Generally, the processor 132 may, for example, provide commands to the stimulation controller 134 to control parameters of the stimulation. The memory 134 may be configured to store programming instructions for the processor 132 to use in providing commands to the stimulation controller 134 to operate the stimulator. The stimulation controller 134 may be configured to generate a stimulation signal (e.g., waveform signal) and deliver the stimulation signal to the stimulator.

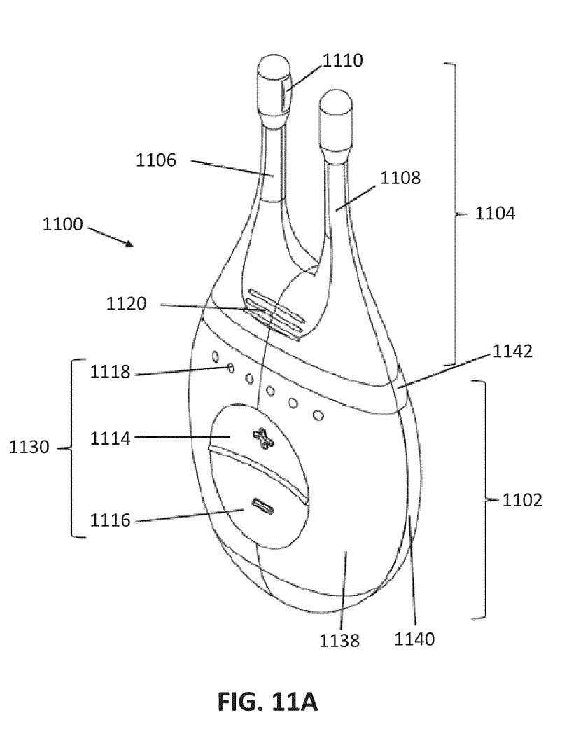

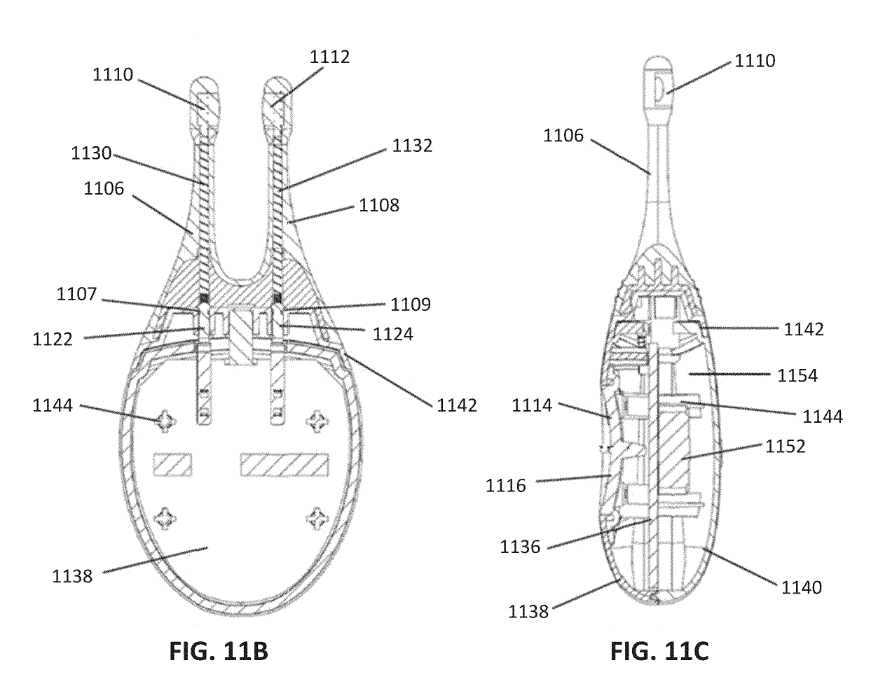

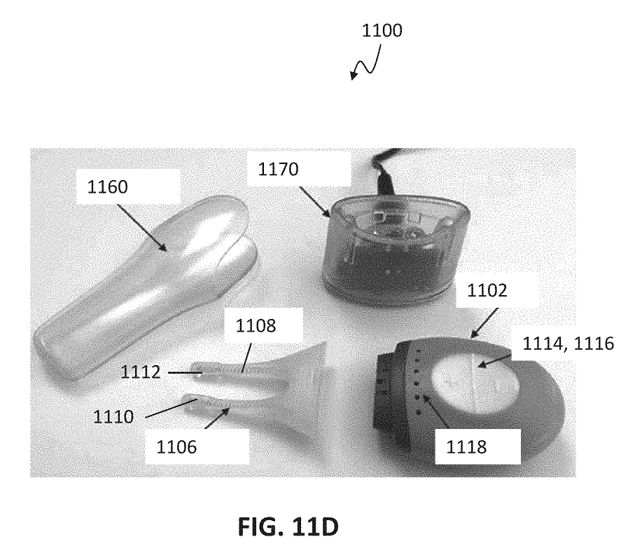

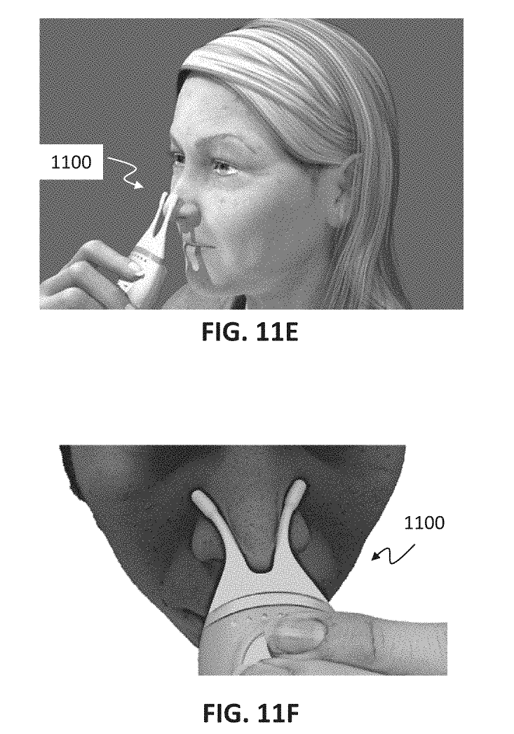

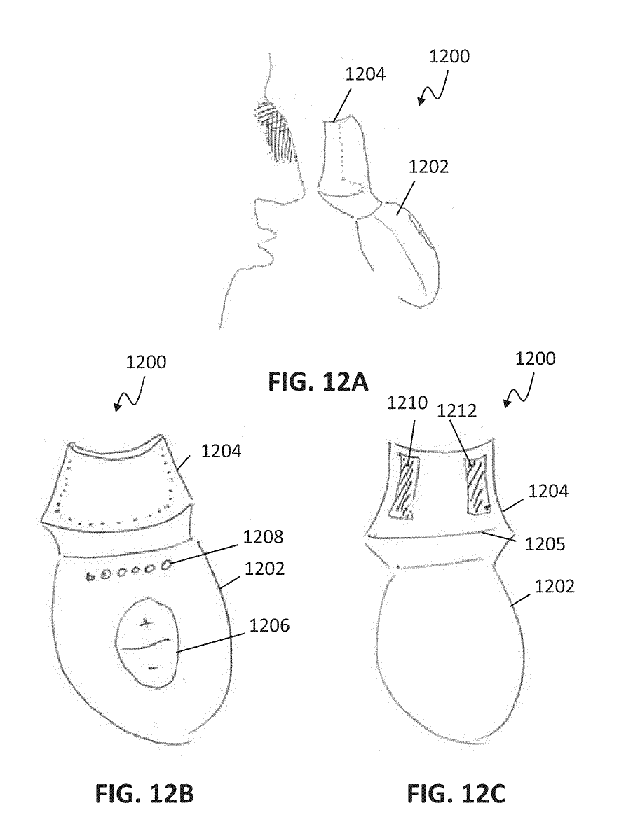

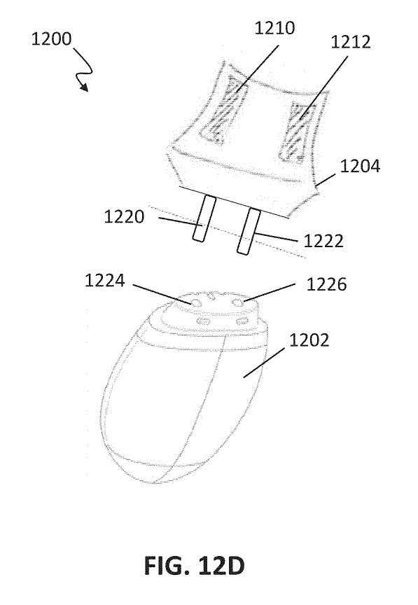

Some or all of the control system 130 may be included in a wearable device (e.g., eyeglasses as shown in FIGS. 3A and 3B) or otherwise proximate the one or more stimulators 110. Additionally or alternatively, some or all of the control system 130 may be located in a handheld unit (e.g., handheld stimulation device as shown in FIGS. 11A-11C and FIGS. 12A-12D) or other suitable device. At least part of the control system 130 may communicate with the stimulators 110 with a wired connection, or with a wireless connection. Furthermore, the control system 130, the stimulators 110, and/or other portions of the stimulation system may be coupled via a wired or wireless connection to a power source, such as a battery.

In some variations, the memory 134 may be configured to store information (e.g., sensor data) that was detected before, during, and/or after stimulation. This information may, for example, be used during execution of programming instructions for adjusting stimulation. Additionally or alternatively, the information may be used to create a medical record of the subject, such as for identifying patterns of the subject's symptoms for various activities, identifying trigger events (e.g., wind as a greater contributor to a particular subject's dry eye symptoms compared to ambient light changes), and the like. Furthermore, the stored information may be used as part of an adaptive learning algorithm, as described below.

Even further, in some variations, the memory 134 (or another storage device) may be configured to store preferences of the subject relating to stimulation. For example, the memory 134 may store one or more preferred stimulation patterns (e.g., preferred intensity that is both comfortable and known to the subject for successfully causing tear production). One or more of the preferred stimulation patterns may be associated with a particular activity or environment, such that the control system triggers a preferred stimulation pattern when the system detects or is notified that the subject is engaging in that activity or in that environment. As another example, the memory 134 may be configured to store one or more stimulation schedules that are preferred by the subject over the course of a typical day (e.g., frequency of treatment sessions).

The memory 134 may store programming instructions for adjusting stimulation based at least in part on the detected characteristics of the subject and/or detected environmental conditions. For example, the control system 130 may be configured to compare the detected characteristics and/or detected environmental conditions, individually and/or in combination, to one or more predetermined thresholds. Such a comparison may involve, for example, a table lookup and/or parametric model, using a table or formula stored in memory 134 or received from another storage medium. Depending on result of the comparison performed, the processor may provide commands to the stimulation controller 134 to provide stimulus signals to operate the one or more stimulators accordingly. For example, the control system 130 may be configured to trigger or increase stimulation (e.g., increase intensity) in response to sensor data generally associated with dry eye symptoms, environmental conditions likely to increase dry eye symptoms, and/or unsuccessful stimulation for tear production.

Additionally or alternatively, in some variations the control system 130 may be configured to adjust stimulation based at least partially on temporal conditions. For example, the control system 130 may be configured to trigger or increase stimulation periodically (e.g., provide stimulation every 10 minutes, every 30 minutes, every hour, etc.). As another example, the control system 130 may be configured to trigger or increase stimulation after the passage of a predetermined amount of time since the occurrence of the most recent stimulation (e.g., provide stimulation if the most recent stimulation occurred 10 minutes ago, 30 minutes ago, an hour ago, etc.), which may or may not have been at least partially based on sensor data gathered as described herein. In yet other example, the control system 130 may be configured to adjust stimulation based on the time of day. In some variations, stimulation during the day (e.g., during business hours) may be different than stimulation during the evening (e.g., after business hours). For example, stimulation during the day may energize or activate a target nerve or other target tissue to a greater extent (e.g., have higher intensity) compared to stimulation during the evening.

In some variations, the control system 130 may be configured to trigger or increase stimulation at least partially based on the occurrence, or in anticipation of, a predetermined event. For example, the control system 130 may be synced to a subject's calendar such that certain kinds of events (e.g., business meeting, air travel, etc.) that may result in increased severity of dry eye symptoms may serve as a basis for adjusting the stimulation for the subject. Such adjustment may, for example, involve triggering stimulation according to stored preferred stimulation patterns (e.g., for particular environments).

As another example, the control system 130 may additionally or alternatively be configured to adjust stimulation based at least in part on one or more adaptive learning algorithms (e.g., machine learning algorithms). For example, in some variations, a predictive model for assessing symptom severity for a particular subject (or set of similar subjects) may be developed based on subject characteristics and/or environmental conditions. Such a predictive model may be trained using empirical training data for the subject (or similar subjects). The predictive model may, for example, be trained using one or more suitable machine learning algorithms (e.g., regularized multi-variate regression algorithm, any suitable supervised or unsupervised machine learning algorithm such as a neural network algorithm, decision tree, etc.). In some variations, the predictive model may be continually updated or trained based on new sensor data from the stimulation device.

In one illustrative example, the control system 130 may be configured to adjust stimulation in accordance with an adaptive learning algorithm that assesses the subject's dry eye severity based on blinking data (e.g., blink frequency, blink duration, blink strength, etc.) and/or eye redness data collected over time (e.g., as the subject wears eyeglasses shown in FIGS. 3A and 3B). As another example, the control system 130 may be configured to adaptively adjust stimulation based at least in part on the time the subject has been awake. Awake time may be estimated, for example, based on detected characteristics such as blinking data, and/or environmental conditions such as duration of sensed ambient light, etc. Based on the awake time, the control system may run a predictive algorithm that assesses when stimulation should be applied in order to minimize dry eye symptoms for the subject. However, it should be understood that in other examples, other characteristics of the subject and/or environmental conditions may provide data for the adaptive learning algorithm to better treat the subject's dry eye and other ocular conditions.

As yet another example, the control system 130 may additionally or alternatively be configured to adjust stimulation at least based on user input via a user interface. One example of a user interface for a stimulation device is shown in FIG. 11A, which depicts handheld stimulation device including a user interface 1130 with buttons 1114 and 1116, which may be manually manipulated to turn stimulation on/off and/or adjust intensity level of the stimulation. However, it should be understood that other variations of stimulation devices (e.g., glasses, masks, etc. described herein) may include a similar user interface providing a subject with manual control of stimulation. For example, a subject may use a push-button or the like, in order to control on-demand stimulation by stimulators on eyeglasses worn by the subject.

Although the sensor and control systems are primarily described herein for use with extranasal stimulation, it should be understood that in other variations, similar sensor arrangements and/or control schemes may be used in combination with other forms of stimulation. For example, stimulation may be provided intranasally. Exemplary intranasal stimulation devices are described in U.S. Pat. No. 8,996,137 titled “NASAL STIMULATION DEVICES AND METHODS” and filed Apr. 18, 2014 which is incorporated herein in its entirety by this reference. As another example, stimulation may be provided via a microstimulator implant implanted intranasally, in an orbit of an eye, or other suitable location. Exemplary microstimulator implants are described in U.S. Pat. No. 9,821,159 titled “STIMULATION DEVICES AND METHODS” and filed Apr. 6, 2012, which is incorporated herein in its entirety by this reference.

### Exemplary Devices

FIGS. 3A and 3B depict an exemplary eyeglasses variation of a stimulation device 300. The device 300 includes two stimulators 310 including electrodes that are configured to deliver an electrical stimulus to facial tissue of the subject when the device 300 is worn by the subject. The device 300 may further include a light sensor 340 configured to detect ambient light and/or a second sensor 342 configured to detect any one or more of direct pressure, humidity, acceleration, and air pressure. A control system 330, located in or coupled to the frame of the device 300, may receive signals from the sensors 340 and/or 342 and adjust the stimulation from the stimulators 310 to treat or preemptively reduce dry eye symptoms based on the received sensor signals.

At least a portion of the eyeglasses may be adjustable to conform or otherwise fit the face of the subject. For example, the bridge of the eyeglasses frame may be adjustable (e.g., flexible plastic or wireframe) to suitably contour the eyeglasses frame to the front of the subject's face. As another example, the arms of the eyeglasses frame may be adjustable to securely couple around the ears of the subject. The lenses of the eyeglasses may, in some variations, provide vision correction (e.g., correction for near-sightedness, far-sightedness, astigmatism, etc.). Alternatively, one or both of the lenses may be blank lenses not providing vision correction (e.g., non-prescriptive).

In some variations, the stimulators 310 may include conductive hydrogel electrode pads disposed on the nose bridge of the eyeglasses, one hydrogel pad for stimulating each side of the subject's nose. The hydrogel pads may be removable from the eyeglasses, such as to be replaceable. Different sizes of electrode pads may be provided so as to accommodate a variety of sizes and shapes of noses. The hydrogel may include any suitable hydrogel, such as those described in U.S. Pat. No. 9,770,583 titled “POLYMER FORMULATIONS FOR NASOLACRIMAL STIMULATION” filed Feb. 24, 2015, which is incorporated herein in its entirety by this reference. In other variations, the stimulators 310 may include other suitable conductive surfaces. In some variations, the electrodes may deliver electrical stimulation as described in U.S. Pat. No. 9,687,652 titled “STIMULATION PATTERNS FOR TREATING DRY EYE” and filed Jul. 24, 2015, which is incorporated herein in its entirety by this reference, or other suitable stimulation.

FIG. 10 depicts an exemplary variation of a stimulation device 1000 that is similar to the stimulation device 300 depicted in FIGS. 3A and 3B, except that the stimulation device 1000 may include an external nasal strip 1002 coupled to the eyeglasses. Alternatively, the external nasal strip 1002 may be standalone. The nasal strip 1002 may be configured to couple to the nose of a subject in any suitable manner. For example, the stimulation device 1000 may include an adhesive backing that allows the nasal strip 1002 to stick to external nasal tissue. As another example, the stimulation device 1000 may include a nasal clip that compresses the nasal tissue in order to couple to the nose of the subject (e.g., a spring-biased clip). As yet another example, the stimulation device 1000 may include at least one hydrogel electrode with a sticky or tacky consistency that, when in contact with external nasal tissue, allows the nasal strip to stick to the nose of the subject. The nasal strip may, in some variations, be made of fabric, flexible plastic, or other suitable material that may, for example, enable the nasal strip to conform to the nose of the subject.

In some variations, the nasal strip 1002 may include one or more stimulators 1010 (e.g., electrodes, ultrasound transducers), including at least one stimulator 1010 disposed on each side of the nasal strip for stimulating a left side and a right side of the subject's nose. For example, the stimulators 1010 may be coupled to a tissue-facing side of the nasal strip 1002 via adhesive, hooks, or any suitable mechanism. The stimulators 1010 may be electrically connected via leads to a power supply and/or a control system on the eyeglasses. In some variations, the stimulators may be configured to stimulate tissue to induce mucin production in the eye, mucous production in the nose, remove old mucous from nose, etc., thereby improving congestion of the subject.

Additionally or alternatively, the nasal strip may be a nasal dilator by pulling on the outside tissue and thereby keeping the breathing passage open and improving nasal congestion. In some variations, the nasal strip and its stimulators may be a standalone device by omitting the eyeglasses. For example, in these variations, instead of connecting to a power supply and/or control system on eyeglasses, a power supply (e.g., battery) and a control system may be disposed on the nasal strip itself. Alternatively, stimulators 1010 on the nasal strip may receive power and/or control signals from a remote control system through wireless communication.

FIG. 4 illustrates an exemplary goggles variation of a stimulation device 400. The stimulation device 400 may include, for example, a housing 402 including an eye shroud or other suitable enclosure, at least one viewing window 404 coupled to the housing, and a strap 406 or other suitable attachment configured to attach the housing 402 to an ocular region of the subject.

The housing 402 may be contoured to complementarily receive the face of the subject. For example, the housing 402 may include an eye shroud that includes a concave profile that conforms to the face of the subject. As another example, the housing 402 may include a flexible eye shroud (e.g., made of rubber or flexible plastic) that flexes to conform to the face of the subject. In some variations, the flexible eye shroud may form a substantially airtight seal against the face of the subject so as to help reduce evaporation of tear film from eyes of the subject.

The stimulation device 400 may additionally include one or more stimulators 410 (e.g., electrodes, ultrasound transducers, etc.) disposed on the interior of the housing and proximate the face of the subject. The stimulators 410 may, for example, line at least a portion of an interior portion of the housing 402 and/or viewing window 404 so as to contact or face the external facial skin of the subject. In some variations, the stimulators 410 contact the skin of the subject around an orbit, so as to stimulate the orbicularis muscle of the subject. As described above, periodic stimulation of the orbicularis muscle may retrain the muscle for rehabilitation and/or regrow the muscle mass for improved muscle strength, such as for easier meibum expression. In some variations, the stimulators 410 may additionally or alternatively improve facial appearance as the eyelids become more healthy-looking in shape and appearance. The stimulators 410 may be controlled by a control system (not shown) which may adjust the stimulation based on sensor data and/or in response to a user input.

The stimulation device 400 may additionally or alternatively include one or more heating sources 412 (e.g., resistive elements, thermal heating gel, etc.) disposed in the housing and configured to be positioned proximate the face of the subject. In some variations, the stimulation 400 may further include one or more cooling sources. Furthermore, the stimulation device 400 may additionally or alternatively include one or more massaging elements 414, such as beads or mechanically actuated projections. The heating sources, cooling sources, and/or massaging elements may be configured to relax the orbicularis muscle and thereby improve meibum expression.

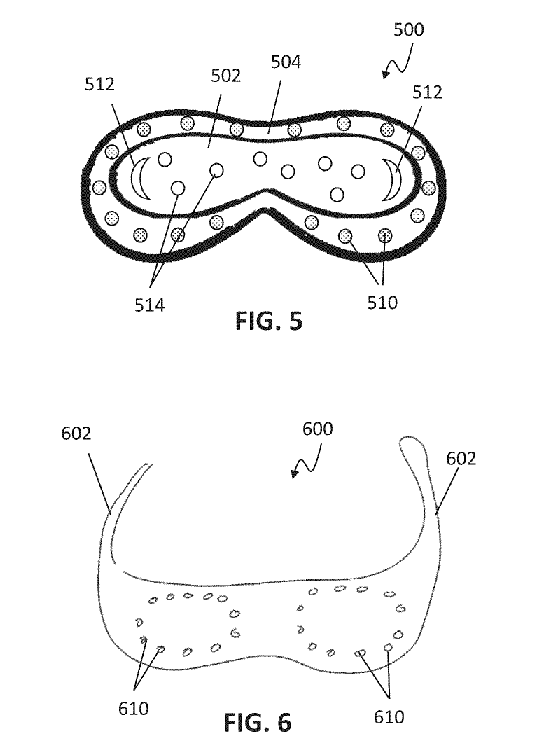

FIG. 5 depicts an exemplary eye mask variation of a stimulation device 500. The stimulation device 500 may be configured to cover at least the ocular region of a subject. For example, the stimulation device 500 may include a strap configured to wrap around the head of the subject, or may include adhesive to attach the stimulation device 500 to the face of the subject. Alternatively, the stimulation device 500 may be configured to simply rest upon the face of the subject (e.g., if the subject is lying down). In some variations, the mask material of the stimulation device 500 may include fabric, flexible plastic, wire skeleton, and/or other suitable flexible material configured to conform to the face of a subject wearing the mask.

Stimulators 510, heating sources 512, cooling sources, and/or massaging elements 514 may be distributed across the stimulation device 500. For example, as shown in FIG. 5, the stimulation device 500 may include a central region 502 and a peripheral region 504. A plurality of stimulators 510 (e.g., electrodes, ultrasound transducers) may be disposed on the peripheral region 504 for stimulating the orbicularis muscle. For example, the stimulators 510 may be arranged in the peripheral region so as to at least partially surround the orbit of the subject when the subject is wearing the stimulation device. The stimulators 410 may, for example, be adhered to a subject-facing surface of the eye mask with adhesive or other suitable mechanism. A plurality of heating sources 512, cooling sources, and/or massaging elements 514 may be disposed on the central region 502. These therapeutic components may, for example, be adhered to a subject-facing surface of the eye mask with adhesive or other suitable mechanism. In some variations, at least some of the heating sources 512, cooling sources, and/or massaging elements 514 may be disposed within enclosures or pockets of the eye mask that help contain the therapeutic elements within the central region.

FIG. 6 depicts another exemplary eye mask variation of a stimulation device 600. Like the stimulation device 500 described above, the stimulation device 600 may be configured to cover at least the ocular region of a subject. Similar to the eye mask described above with respect to FIG. 5, the eye mask shown in FIG. 6 may include fabric, flexible plastic, wire skeleton, and/or other suitable flexible material configured to conform to the face of a subject wearing the mask. The eye mask may include straps 602 configured to attach the stimulation device 600 to the face of the subject. The stimulation device 600 may include a plurality of stimulators 610 (e.g., electrodes, ultrasound transducers, etc.) for stimulating around the rim of the skull near the eye (e.g., around the orbit), for stimulating one or more branches of the ophthalmic nerve. For example, the stimulators 610 may be arranged in at least a partial ring so as to at least partially surround the orbit of the subject when the subject is wearing the stimulation device 600. The stimulators 610 may, for example, stimulate one or branches of the ophthalmic nerve of the user. Additionally or alternatively, the stimulation may provide relaxation (e.g., at night). Furthermore, the mask may cover the ocular region so as to reduce evaporation of tear film (existing and/or freshly stimulated tears).

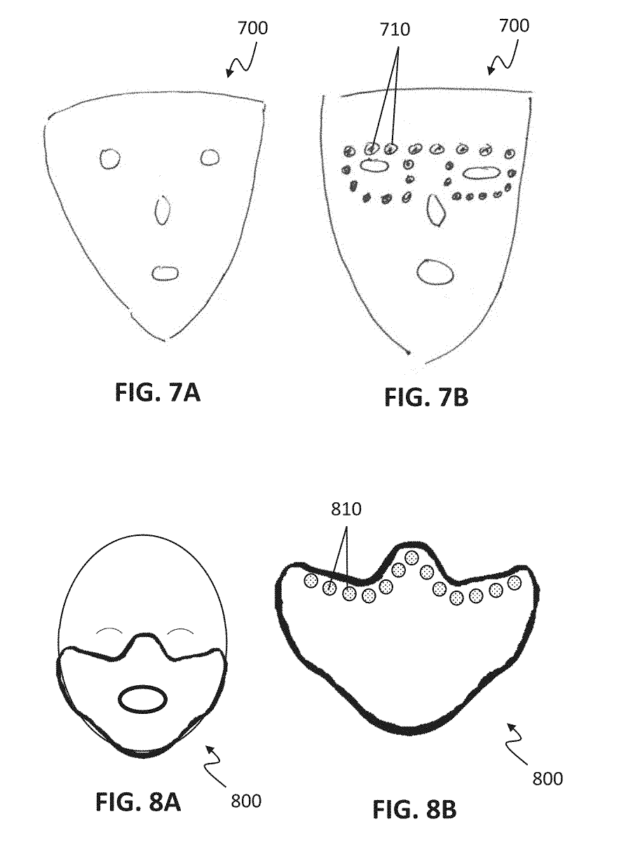

FIGS. 7A and 7B depict front and rear sides, respectively, of an exemplary full face mask variation of a stimulation device 700. The stimulation device 700 may be similar to the stimulation device 500 described above, except that the stimulation device 700 may be configured to substantially cover the entire face of the subject. For example, the stimulation device 700 may include a plurality of stimulators 710 (e.g., electrodes, ultrasound transducers, etc.) arranged in at least a partial ring and positioned to at least partially surround the orbit of the subject when the subject is wearing the stimulation device 700, for stimulating the orbicularis muscle. Furthermore, at least some of the stimulators 710 may be arranged on the mask so as to stimulate one or more nerve targets described herein (e.g., infratrochlear nerve), thereby increasing tear production. Similar to the eye mask described above with respect to FIG. 5, the mask shown in FIGS. 7A and 7B may include fabric, flexible plastic, wire skeleton, and/or other suitable flexible material configured to conform to the face of a subject wearing the mask.

FIGS. 8A and 8B depict front and rear sides, respectively, of another exemplary mask variation of a stimulation device 800. The stimulation device 800 may include a plurality of stimulators 810 (e.g., electrodes, ultrasound transducers, etc.) arranged along the rim of a mask configured to contact the cheekbones (near the orbit) of the subject. For example, as shown in FIG. 8B, the stimulators 810 may be arranged along a superior edge of the mask, such that when the subject is wearing the mask, the stimulators 810 may be configured to stimulate one or more branches of the ophthalmic nerve (CN VI branches). Additionally or alternatively, the stimulation may provide relaxation (e.g., at night) using modulation effects on the sympathetic and parasympathetic nervous system. For example, stimulation may be configured to induce alpha waves (e.g., between about 7 Hz and 13 Hz) and/or theta waves (e.g., between about 4 Hz and 7 Hz). Suitable stimulation parameters may be determined, for example, by modulating stimulation delivered to the subject or a test subject, and its effects reviewed and verified via fMRI and/or other suitable manners. Similar to the eye mask described above with respect to FIG. 5, the mask shown in FIGS. 8A and 8B may include fabric, flexible plastic, wire skeleton, and/or other suitable flexible material configured to conform to the face of a subject wearing the mask.

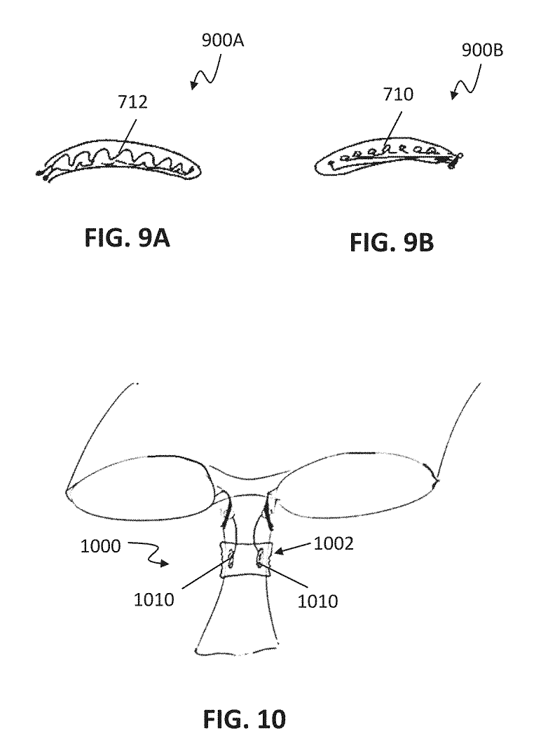

FIGS. 9A and 9B depict exemplary stimulation devices including pads configured to be coupled to the eyelids of the subject, such as near the rim of the eyelid. The pads may be contoured and/or conformable to correspond to the shape of an eyelid. The devices 900A and 900B may be configured to couple to eyelid skin in any suitable manner. For example, the devices 900A (FIG. 9A) and 900B (FIG. 9B) may include an adhesive backing that allows the pads to stick to the eyelid skin. As another example, one or both of the devices 900A and 900B may include at least one hydrogel electrode with a sticky or tacky consistency that, when in contact with the eyelid skin, allows the pad to stick to the eyelid skin.

In some variations, as shown in FIG. 9A, a stimulation device 900A may include one or more heating sources 712 (e.g., one or more resistive elements, heating gel, etc.). For example, the heating sources 712 may be distributed along the entire length of the pad. In other variations, as shown in FIG. 9B, a stimulation device 900B may include one or more stimulators 710 (e.g., electrodes, ultrasound transducers, etc.) configured to deliver stimulation to an eyelid when the device 900B is coupled to the eyelid. For example, the electrodes 710 may be distributed along the entire length of the pad. The pads may, for example, be controlled with a control system (not shown) that is in communication with the stimulators via a wireless connection. Alternatively, the stimulators may be coupled to leads for wired control of the stimulation. The pads 900A and 900B may provide efferent stimulation and/or afferent stimulation of CN VI nerves innervating the eyelids. In some variations, the stimulation target by the stimulators may be selected to trigger either more efferent activity or more afferent activity.

FIGS. 11A-11C depict perspective, cut-away rear, and cut-away side views, respectively, of an exemplary handheld variation of a stimulation device 1100. The stimulation device 1100 may include a handheld body 1102, at least one projection (shown in a projection region 1104 comprising prongs 1106 and 1108) coupled to the handheld body 1102, and at least one stimulator (shown as stimulators 1110 and 1112) coupled to the one or more projections and configured to deliver a stimulus.

As shown in FIG. 11C, the handheld body 1102 may comprise a front housing 1138, a back housing 1140, and/or a proximal housing 1142, which may fit together to define a body cavity 1154. For example, as shown in FIGS. 11B and 11C, one or more fasteners 1144 may couple the front housing 1138 and the back housing 1142 together. The proximal housing 1142 may couple to the front and back housings (e.g., via a snap fit). However, the front housing 1138, the back housing 1140, and/or the proximal housing 1142 may couple in any suitable manner. The body cavity 1154 may contain a control system 1136 (e.g., comprising one or more processors) and a power source 1152 (e.g., battery), which together may generate and control the stimulus. In some variations, the power source 1152 may be recharged (e.g., via electrical contacts, wireless power transfer, etc.) with a charge station such as the charge station base 1170 shown in FIG. 11D. The charge station base 1170 may include a mount configured to receive the body 1102 and may be connected to a power outlet or other suitable power source.

The stimulus may be delivered to a subject via the projection region 1104. In some variations the body 1102 and projection region 1104 may be reversibly attachable. Some or all of the stimulator 1100 may be disposable, and some or all of the stimulator 1100 may be reusable. For example, in variations where the projection region 1104 is releasably connected to the body 1102, the body 1102 may be reusable, and the projection region 1104 may be disposable and periodically replaced. In some of these variations, the device may include a disabling mechanism that prevents stimulus delivery to the subject when the projection region 1104 is reconnected to the body after being disconnected from the body. Additionally or alternatively, the device may include a lockout mechanism that prevents the projection region 1104 from being reconnected to the stimulator body after being disconnected from the stimulator body. In some variations, as shown in FIG. 11D, the device further comprises a detachable protective cap 1160 that may reversibly attach to the projection region 1104 and/or body 1102 (e.g., via snap fit engagement) so as to temporarily cover the projection region 1104, such as for protective storage.

The projection region 1104 may include at least one prong 1106, which may be configured to be placed in contact with, or proximate to, external facial tissue of the subject. In the handheld stimulator variation shown in FIGS. 11A-11C, the projection region 1104 may include two prongs 1106 and 1108. At least a portion of the projection region 1104 may conform to the external facial tissue of the subject. For example, one or both of the prongs 1106 and 1108 may be flexible and/or conform at least partially to extranasal tissue as shown in FIGS. 11E and 11F. In this example, the subject may hold the device 1100 against his or her nose such that tips of the prongs 1106 and 1108 contact and flex or bend against opposing sides of his or her nose. As another example, one or both prongs 1106 and 1108 may have a curved shape (e.g., laterally outwardly curving shape) or other suitable shape to better position the stimulators at target tissue regions of interest. In some variations, the prongs 1106 and 1108 may include a flexible material (e.g., pliable plastic, silicone, other suitable rubber, etc.) and/or articulatable joints to allow the projection region 1104 to conform to the external facial tissue of the subject.

In some variations, the projection region 1104 may include an alignment feature to help the subject appropriately position the prongs 1106 and 1108 on his or her external facial tissue. For example, the projection region 1104 may include a stop (e.g., located between the prongs) configured to abut a distal tip of the subject's nose. Alignment of the stop against the distal tip of the subject's nose may help appropriately position stimulators 1110 and 1112 against a target region for stimulation. In some variations, as shown in FIG. 11A, the projection region 1104 may further comprise ridges 1120, which may allow the subject to more easily grip the projection region 1104.

The projection region 1104 may comprise at least one stimulator (e.g., electrode, ultrasound transducer, etc.). For example, as shown in FIGS. 11A-11C, the projection region 1104 may comprise a first stimulator 1110 on a first prong 1106 and a second stimulator 1112 on a second prong 1108. In some variations, the stimulators 1110 and 1112 on the prongs 1106 and 1108, respectively, may be configured to stimulate the exit point of the anterior ethmoidal nerve on the outside of the nose, and/or stimulate the infratrochlear nerve on the nose and/or facial anatomy near the eyes. Other suitable targets may be stimulated by the stimulators 1110 and 1112.

As shown in the cut-away view of the stimulator 1100 in FIG. 11B, the electrodes 1110 and 1112 may be connected to leads 1130 and 1132 located within prongs 1106 and 1108, respectively. The leads 1130 and 1132 may in turn be connected to connectors 1122 and 1124, respectively. Connectors 1122 and 1124 (e.g., male connector pins) may extend through lumens 1107 and 1109 in the proximal housing 1142, and may connect directly or indirectly to the control subsystem 1136 and/or power source 1152. As such, a stimulus may travel from the control subsystem 1136 through the connectors 1122 and 1124, through the leads 1130 and 1132, and through the stimulators 1110 and 1112. Alternatively, the body 1102 may include connectors that extend through lumens in the prongs 1106 and 1108. In some variations, the stimulators 1110 and 1112 comprise a hydrogel electrode, which is described in more detail in U.S. Pat. No. 9,770,583 titled “POLYMER FORMULATIONS FOR NASOLACRIMAL STIMULATION” filed Feb. 24, 2015, which was previously incorporated in its entirety.

The body may comprise a user interface 1130 comprising one or more operating mechanisms to adjust one or more parameters of the stimulus. The operating mechanisms may provide information to the control subsystem 1136, which may comprise a processor, memory, and/or stimulation controller as described above. In some variations, the operating mechanisms may comprise first and second buttons, as illustrated for example in FIGS. 11A and 11C as 1114 and 1116. In some variations, pressing the first button may turn on the stimulator and/or change the stimulus waveform, while pressing the second button 1116 may turn off the stimulator and/or change the stimulus waveform. Additionally or alternatively, the user interface 1130 may comprise one or more feedback elements (e.g., based on light, sound, vibration, or the like). As shown, the user feedback elements may comprise light-based indicators (e.g., LED), shown in the variation of FIG. 11A as one or more indicators 1118, which may provide information to the user. This stimulator and other hand-held stimulators that may deliver the electrical stimuli described herein are described in U.S. Pat. No. 9,687,652, which was previously incorporated by reference in its entirety.

FIGS. 12A-12C depict a side view, a front view, and a rear view of another exemplary handheld variation of a stimulation device 1200. The stimulation device 1200 may be similar to the stimulation device 1100 described above with reference to FIGS. 11A-11D, except as described below. Like the stimulation device 1100, the stimulation device 1200 may include a handheld body 1202, at least one projection 1204, and at least one stimulator (shown as stimulators 1210 and 1212 in FIG. 12C) coupled to the projection 1204 and configured to deliver a stimulus. However, in the variation shown in FIGS. 12A-12C, the projection 1204 may include a concave surface configured to conform to external facial tissue of the subject, such as a nose of the subject. The device 1200 may include stimulators 1210 and 1212 (e.g., electrodes, ultrasound transducers) coupled to a tissue-facing side of the concave surface. In this variation, the first and second stimulators 1210 and 1212 may be disposed on opposing sides of the projection 1204, such that the stimulators are configured to be in contact with or proximate opposing sides of the subject's nose when the projection 1204 is held against the subject's face. Although only two stimulators 1210 and 1212 are shown in FIG. 12C, it should be understood that in other variations, fewer (e.g., one stimulator) or more (e.g., three, four, five, six, or more) stimulators may be disposed on the projection 1204. In some variations, various sizes of the projection 1204 may be provided, and the suitable size (e.g., small, medium, large, etc.) of the projection 1204 may be selected to fit a particular subject. Other characteristics, such as radius of curvature and/or depth of the projection 1204 (e.g., shallow for accommodating a flat nose, deep for accommodating a longer prominent nose, etc.), may vary among different sizes of projections 1204, and the appropriate size/shape of the projection 1204 may be similarly selected to fit a particular subject.

As shown in FIGS. 12A-12C, the concave surface of the projection 1204 may have a curved or general cup shape generally configured to receive a nose of the subject. The projection 1204 may include a flexible material, such as a thin pliable plastic or rubber (e.g., silicone), that may be flex or bent to conform to the external facial tissue of the subject. Additionally or alternatively, the projection 1204 may include pleats, hinges, sliding plates, or other articulable joints to allow the projection region 1204 to conform to the external facial tissue of the subject.

As shown in FIG. 12D, the projection 1204 may be reversibly attached to the handheld body 1202. For example, the projection 1204 may have a surface that complementarily mates with a corresponding surface on the body 1202 (e.g., via snap fit or other suitable fasteners). Furthermore, similar to the device 11000 described above, the stimulators 1210 and 1212 may be connected to one or more leads (not shown). These leads may in turn be connected to connectors 1220 and 1222 (e.g., male connector pins), respectively. Connectors 1220 and 1222 may extend through lumens 1224 and 1226 in the body 1202 and may connect directly or indirectly to a control subsystem and/or power source disposed within the body 1202. Alternatively, the body 1202 may include connectors that extend through lumens in the projection 1204.

In some variations, the projection 1204 may include an alignment feature to help the subject appropriately position the projection 1204 on his or her external facial tissue. For example, the projection region 1204 may include a stop such as a ridge 1205 located along the bottom of the projection 1204 and configured to abut a distal tip of the subject's nose. Alignment of the stop against the distal tip of the subject's nose may help appropriately position stimulators 1110 and 1112 against a target region for stimulation.

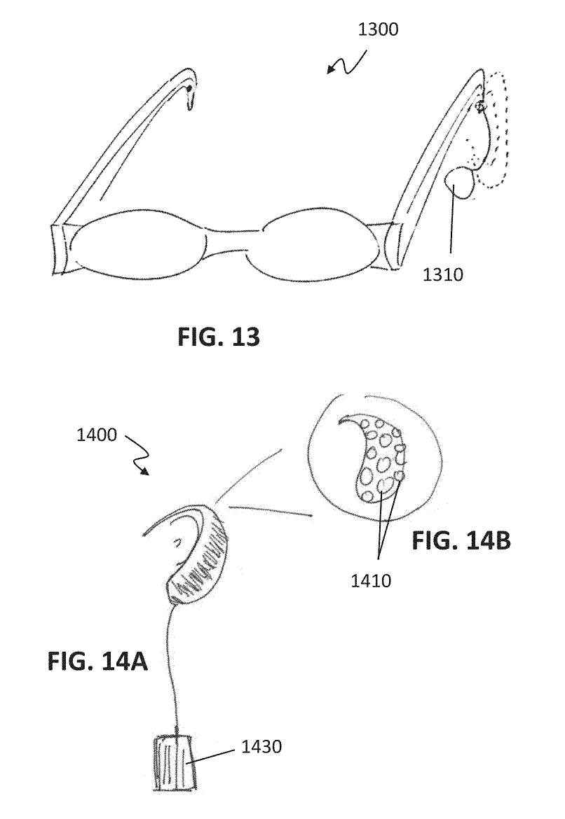

FIG. 13 depicts another exemplary eyeglasses variation of a stimulation device 1300 including one or more stimulators 1310 configured to stimulate a subject in a region between the subject's nose and ear. For example, the stimulator 1310 (e.g., electrode, ultrasound transducer, etc.) may be configured to couple to the skin of the subject near an exit point of the facial nerve (CN VII) via adhesive and/or hydrogel electrodes, in a manner similar to that described above with reference to FIGS. 9A and 9B. The stimulators 1310 may be electrically connected via leads to a power supply and/or a control system on the eyeglasses, such as on the arm of the eyeglasses or other suitable portion of the frame. Alternatively, the stimulators may be communicatively coupled in a wireless fashion to receive power from a power supply and/or to receive control signals from a wireless control system. Additionally or alternatively, the stimulators 1310 may be disposed directly on the frame of the eyeglasses, such that they contact the subject at a target region when the subject wars the eyeglasses.

FIGS. 14A and 14B depict an exemplary over-the-ear variation of a stimulation device 1400 including a plurality of stimulators 1410 that are configured to contact skin of the ear when the device 1400 is worn by the subject. As shown in the overall view of FIG. 14A, the stimulation device 1400 may include, for example, a clip that couples to the pinna of the subject's ear. In some variations, the clip may be contoured around the pinna so as to mate with the pinna. For example, the clip may include a longitudinal, curved channel configured to receive the pinna of the subject's ear. Additionally or alternatively, the clip may include spring-biased members, opposing tabs, or other suitable clamping mechanism that allow the stimulation device 1400 to clamp onto the pinna. In yet other variations, the clip may additionally or alternatively include rubber or other higher friction materials, textural patterns (e.g., ribs, raised dots, etc.) on the surface configured to contact the skin of the subject, in order to enable the clip to better grip and couple to the pinna. In yet other variations, the clip may attach to the subject via adhesive and/or hydrogel electrodes in a manner similar to that described above with reference to FIGS. 9A and 9B.

As shown in FIG. 14B, a plurality of stimulators 1410 (e.g., electrodes, ultrasound transducers, etc.) may be disposed on an underside or interior of the clip (that is, the subject-facing surface). In some variations, for example, one or more of the stimulators may be configured to stimulate one or more nerves in the preauricula, mastoid, lobule, and/or helix regions, including but not limited to the auricular branch of the vagus nerve, the auriculotemporal nerve, and the greater auricular nerve. The stimulators 1410 may be electrically connected via leads to a power supply and/or a control system on the clip or other suitable structural support (e.g., eyeglasses). Alternatively, the stimulators may be communicatively coupled in a wireless fashion to receive power from a power supply and/or to receive control signals from a wireless control system.

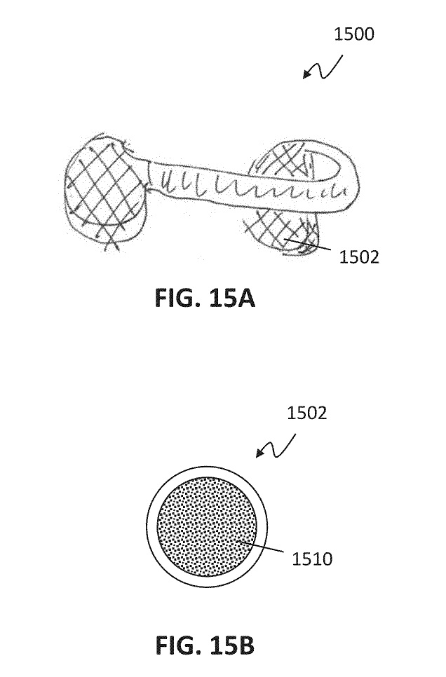

FIGS. 15A and 15B depict an exemplary earmuff variation of a stimulator device 1500. As shown in the overall view of FIG. 15A, the stimulator device 1500 may include pads 1502 configured to cover the ears of a subject wearing the earmuffs, and a band 1504, strap, or other member connecting the pads 1502 and assisting the attachment of the earmuffs to the head of a subject. In some variations, the band may be oriented as shown in FIG. 15A so as to wrap around the back of the subject's head. Alternatively, the band 1504 may be oriented orthogonally to the orientation shown in FIG. 15B, so as to wrap around the top of the subject's head. The band 1504 may be adjustable, such as by including sliding members to adjust band length and/or elastic material or a pleated member to facilitate adjustment in band length.

As shown in FIG. 15B, the pads 1502 may include, on an interior subject-facing surface, a plurality of stimulators 1510 (e.g., electrodes, ultrasound transducers, etc.). The stimulators may, for example, by coupled to fabric of the pads 1502 via epoxy, clips, etc. In some variations, the pads 150 may include conductive fabric that is suitable to function as an electrical stimulator. Aspects of the over-the-ear variation shown in FIGS. 14A and 14B may be combined with aspects of the earmuff variation shown in FIGS. 15A and 15B. For example, in some variations, the pads 1502 may include an over-the-ear, contoured channel configured to receive the pinna of the subject's ear, or other suitable over-the-ear clip.

Similar to the stimulators 1410 described above with reference to FIGS. 14A and 14B, the stimulators 1510 may be electrically connected via leads to a power supply and/or a control system on the pad 1502, the band 1504, or other suitable structural support (e.g., eyeglasses). Alternatively, the stimulators may be communicatively coupled in a wireless fashion to receive power from a power supply and/or to receive control signals from a wireless control system.

### Stimulation Methods

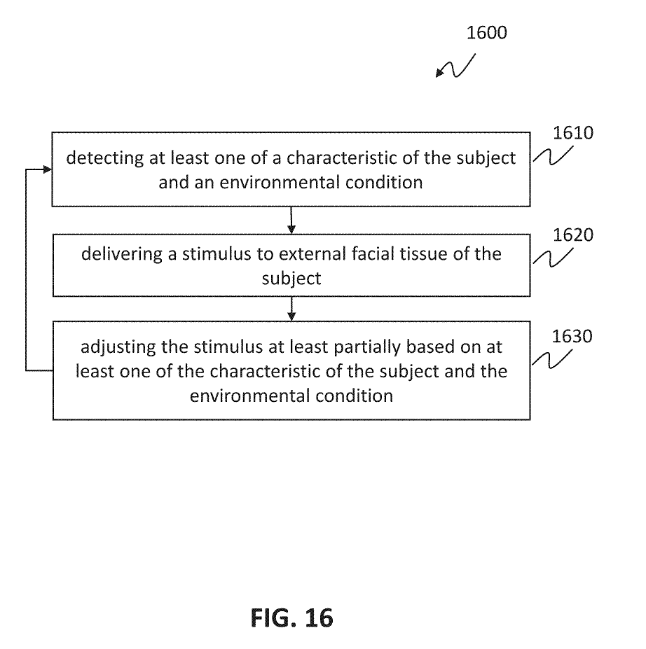

Generally, in some variations, as shown in FIG. 16, a stimulation method 1600 for treating a condition of a subject may include delivering a stimulus 1620 to external facial tissue of the subject, and adjusting the stimulus at least partially based on at least one of a characteristic of the subject and an environmental condition 1630. The stimulus 1620 (e.g., electrical stimulation, ultrasound stimulation, etc.) may be delivered with a stimulation system such as any of the stimulation devices described herein. Furthermore, the stimulus may be delivered such that the stimulus activates a nerve of the subject, as described elsewhere herein. For example, the stimulus may be configured to activate a nerve of the subject to thereby increase tear production (and/or production of a tear component, etc.). As another example, the stimulus may be configured to strengthen one or more muscles (e.g., orbicularis muscle) to help increase tear production and/or production of a tear component.

In some variations, the method 1600 may include detecting at least one of a characteristic of the subject and an environmental condition 1610. The detected subject characteristic and/or environmental condition may serve as a basis for adjusting the stimulus. For example, suitable characteristics of a subject include one or more symptoms of dry eye (e.g., blink rate, blink duration, blink strength, eye redness, tear meniscus height, temperature, etc.), and/or one or more indications of successful stimulation for tear production. Sensors such as image sensors and electromyography sensors may be used to detect (e.g., measure) such subject characteristics. Suitable environmental conditions to serve as a basis for adjusting the stimulus include, for example, ambient light, humidity, wind conditions, air pressure, and other environmental conditions described above. The method may include triggering onset of stimulation (e.g., stimulation in accordance with a stimulation intensity preferred by the subject) or otherwise adjusting stimulation (e.g., increasing intensity of stimulation) in response to detecting one or more dry eye symptoms and/or detecting one or more environmental conditions that may cause and/or exacerbate dry eye symptoms.

In some variations, adjusting the stimulus 1630 may be performed according to an adaptive learning algorithm. For example, as described above, in some variations, a predictive model for assessing symptom severity for a particular subject (or set of similar subjects) may be developed based on subject characteristics and/or environmental conditions. Such a predictive model may be trained using empirical training data for the subject (or similar subjects). The predictive model may, for example, be trained using one or more suitable machine learning algorithms (e.g., regularized multi-variate regression algorithm, any suitable supervised or unsupervised machine learning algorithm such as a neural network algorithm, decision tree, etc.). In some variations, the predictive model may be continually updated or trained based on new sensor data from the stimulation device.

In one illustrative example, the method 1600 may include adjusting stimulation in accordance with an adaptive learning algorithm that assesses the subject's dry eye severity based on blinking data (e.g., blink frequency, blink duration, blink strength, etc.) and/or eye redness data collected over time (e.g., as the subject wears eyeglasses as shown in FIGS. 3A and 3B). As another example, the method may include adjusting stimulation based at least in part on the time the subject has been awake. Awake time may be estimated, for example, based on detected characteristics such as blinking data, and/or environmental conditions such as duration of sensed ambient light, etc. Based on the awake time, a predictive algorithm may assess when stimulation should be applied in order to minimize dry eye symptoms for the subject. However, it should be understood that in other examples, other characteristics of the subject and/or environmental conditions may provide data for the adaptive learning algorithm to better treat the subject's dry eye and other ocular conditions.

### EXAMPLES

### Example 1

Stimulation was performed using a handheld stimulator as shown in FIG. 12C to apply electrical stimulation extranasally. The stimulator was configured to generate an electrical stimulus to be delivered to the subject via hydrogel electrodes on each of two prongs. Observation of the eyelid margin during stimulation showed meibum expression during delivery of extranasal stimulation, as well as tearing (production of the lacrimal aqueous component) during delivery of extranasal stimulation.

### Example 2

Participants were subjected to extranasal stimulation for three minutes using a handheld stimulator shown in FIG. 16. The stimulator was capable of delivering five stimulation intensity levels, as described in more detail in U.S. Pat. No. 9,687,652 titled “STIMULATION PATTERNS FOR TREATING DRY EYE” and filed Jul. 24, 2015, which is incorporated herein in its entirety by this reference. Participants adjusted the level by pressing the plus or minus button to obtain the best tingling “sneezy” sensation. As shown in FIG. 12A, the stimulator system consisted of four distinct parts: 1) A reusable base unit, which produces the electrical stimulation waveform, 2) A disposable tip assembly that inserts into the nasal cavity and stimulates the target intranasal tissue, 3) A reusable cover to protect the tip assembly, 4) A charger, which recharges the battery inside the base unit.

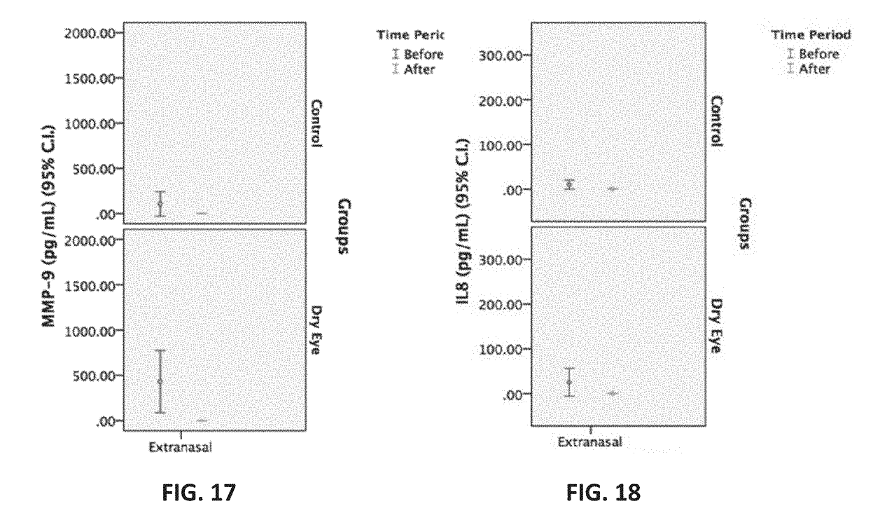

The participants were instructed to place the tips of the handheld stimulator on the lower part of the nose (one tip on each side) for extranasal stimulation, as shown in FIG. 12B. Tear fluid was collected before and after each extranasal stimulation. Tear samples were collected using a microcapillary tube from the tear lake near the temporal canthus without touching the globe. Tear collection continued until a maximum of 5 μL was collected from each eye, or until 5 minutes had elapsed. The tear samples were analyzed for a variety of factors and inflammatory mediators. Although not statistically significant, there was a decrease in MMP-9 and IL-8 concentrations in the tear samples after extranasal application of stimulation. MMP-9 concentration data from subjects who revealed a decrease compared to controls after extranasal stimulation application are provided in the table below and in FIG. 17.

MMP-9 (pg/mL)

| Control (n:2) | | Dry Eye (n:3) | |
|---|---|---|---|
| before | after | before | after |
| extranasal | extranasal | extranasal | extranasal |
| stimulation | stimulation | stimulation | stimulation |
| 267.34 | 0.6 | 1435.71 | 0.6 |

Data illustrating the decrease in IL-8 concentration as compared to controls after extranasal stimulation application are provided in FIG. 18.

### Example 3

Participants included 48 adults with mild to severe dry eye disease, baseline Ocular Surface Disease Index® ≥13, Schirmer test (with anesthesia) ≤10 mm and cotton swab nasal stimulation Schirmer test ≥7 mm higher in the same eye. As part of the study, participants were subjected to extranasal stimulation for three minutes using a handheld stimulator as shown in FIG. 16 and as described in more detail in U.S. application Ser. No. 14/256,915, filed Apr. 18, 2014, and titled “NASAL STIMULATION DEVICES AND METHODS,” which was previously incorporated by reference in its entirety. Mean (SD) Schirmer score for the extranasal stimulation was 9.5 [8.2] mm, while the mean (SD) Schirmer score for sham intranasal stimulation was 9.2 [7.3] mm.

### Example 4

Participants included 25 dry eye subjects. As part of the study, tear meniscus height was measured prior to and immediately following about 2 minutes of extranasal stimulation using a handheld stimulator as shown in FIG. 16 and as described in more detail in U.S. application Ser. No. 14/256,915, filed Apr. 18, 2014, and titled “NASAL STIMULATION DEVICES AND METHODS,” which was previously incorporated by reference in its entirety. The mean change in tear meniscus height (post-minus pre-stimulation) for the extranasal stimulation was 56±198 μm.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A system for treating a condition of a subject, comprising:
a stimulator configured to deliver a stimulus to external facial tissue of the subject such that the stimulus activates a nerve of the subject, thereby increasing tear production;
at least one sensor configured to detect a characteristic of the subject; and
a control system in communication with the sensor and configured to adjust the stimulus at least partially based on the detected characteristic of the subject.

2. The system of claim 1, wherein the stimulus comprises electrical stimulation.

3. The system of claim 1, wherein the stimulus comprises ultrasound stimulation.

4. The system of claim 1, wherein the sensor comprises an image sensor configured to image an ocular region of the subject.

5. The system of claim 1, wherein the sensor comprises an electromyography sensor configured to detect facial muscle contractions.

6. The system of claim 1, wherein the sensor is configured to detect one or more symptoms of dry eye.

7. The system of claim 6, wherein the sensor is configured to detect at least one of blink rate, blink duration, and blink strength.

8. The system of claim 6, wherein the sensor is configured to detect eye redness.

9. The system of claim 6, wherein the sensor is configured to detect tear meniscus height.

10. The system of claim 6, wherein the sensor is configured to measure temperature of an ocular region of the subject.

11. The system of claim 1, wherein the sensor is configured to detect an indication of successful stimulation for tear production.

12. The system of claim 1, further comprising a second sensor configured to detect an environmental condition, wherein the control system is further configured to adjust the stimulus at least partially based on the detected environmental condition.

13. The system of claim 12, wherein the second sensor is configured to measure ambient light.

14. A stimulation method for treating a condition of a subject, comprising:
delivering a stimulus to external facial tissue of the subject such that the stimulus activates a nerve of the subject, thereby increasing tear production; and
adjusting the stimulus at least partially based on a detected characteristic of the subject.

15. The method of claim 14, wherein the stimulus comprises electrical stimulation.

16. The method of claim 14, wherein the stimulus comprises ultrasound stimulation.

17. The method of claim 14, wherein further comprises adjusting the stimulus in response to input of the subject.

18. The method of claim 14, wherein the characteristic of the subject comprises one or more symptoms of dry eye.

19. The method of claim 14, wherein the characteristic of the subject comprises an indication of successful stimulation for tear production.

20. The method of claim 14, further comprising adjusting the stimulus at least partially based on a detected environmental condition.

21-39. (canceled)
